SEQUENCE LISTING (41 sequences)

SEQ ID NO: 1 - DNA, 2433 bp, Homo sapiens
atggaacacaaagaagtggtgttgctcctgctgctgttcctgaagtccggccagggcgagcccctggacgattacgtgaacacccagggcgccagcctgttcagcgtgaccaagaaacagctgggagccggcagcatcgaggaatgcgccgccaagtgcgaagaggacgaggaattcacctgtcgggccttccagtaccacagcaaagaacagcagtgcgtgatcatggccgagaacagaaagagcagcatcatcatcagaatgcgggacgtggtgctgttcgagaagaaggtgtacctgagcgagtgcaagaccggcaacggcaagaactaccggggcaccatgagcaagaccaagaacggcatcacctgtcagaagtggtccagcaccagcccccaccggcctagattttctccagccacccaccctagcgagggcctggaagagaactactgccggaaccccgacaacgaccctcagggcccttggtgctacaccaccgaccccgagaagagatacgactactgcgacatcctggaatgtgaagaggaatgcatgcactgcagcggcgagaactacgacggcaagatctccaagaccatgagcggcctggaatgccaggcttgggacagccagtctcctcacgcccacggctacatccccagcaagttccccaacaagaacctgaagaagaattactgcagaaaccctgaccgcgagctgcggccctggtgttttaccaccgatcctaacaagagatgggagctgtgcgatatcccccggtgcaccacacctccacctagcagcggccctacctaccagtgtctgaagggcaccggcgagaattacaggggcaacgtggccgtgaccgtgtccggccatacctgccagcattggagcgcccagaccccccacacccacaacagaacccccgagaacttcccctgcaagaatctggacgagaattattgtcgcaaccccgatggcaagagggccccctggtgtcacaccaccaacagccaggtgcgctgggagtactgcaagatccccagctgcgatagcagccccgtgtccacagaacagctggcccctacagcccctcctgagctgacacctgtggtgcaggattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 2 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 3 - DNA, 1209 bp, Artificial Sequence
  Exemplary nucleic acid sequence of recombinant PAI-1, Q197C, G355C
atgcagatgtctcccgccctgacctgcctggtgctgggcctggccctggtgttcggagagggctctgccgtgcaccacccacctagctacgtggcacacctggcctccgacttcggcgtgagggtgtttcagcaggtggcccaggccagcaaggatcgcaacgtggtgttcagcccttatggcgtggcctccgtgctggccatgctccagctgaccacaggaggagagacccagcagcagatccaggcagctatgggcttcaagatcgacgataagggaatggcacccgccctgaggcacctgtacaaggagctgatgggcccttggaataaggacgagatcagcaccacagatgccatctttgtgcagcgcgacctgaagctggtgcagggcttcatgccacacttctttcggctgttccggagcaccgtgaagcaggtggacttcagcgaggtggagagggcccgctttatcatcaacgattgggtgaagacccacacaaagggcatgatcagcaatctgctgggcaagggagcagtggatcagctgaccaggctggtgctggtgaacgccctgtacttcaatggctgctggaagaccccatttcccgacagctccacacaccggagactgttccacaagtccgatggctctacagtgagcgtgcctatgatggcccagaccaacaagttcaattatacagagtttaccacacctgacggccactactatgacatcctggagctgccataccacggcgacaccctgagcatgtttatcgccgccccttatgagaaggaggtgccactgtccgccctgacaaacatcctgtccgcccagctgatctctcactggaagggcaatatgaccaggctgccaaggctgctggtgctgcctaagttctccctggagacagaggtggacctgcggaagcctctggagaacctgggcatgaccgatatgttcagacagtttcaggccgactttacatctctgagcgatcaggagccactgcacgtggcacaggccctccagaaggtgaagatcgaggtgaacgagtcctgtaccgtggcctctagctccacagccgtgatcgtgtctgccaggatggccccagaggagatcatcatggatcggcccttcctgtttgtggtgagacacaatccaaccggcacagtgctgttcatgggccaggtcatggagccctga

SEQ ID NO: 4 - protein, 402 aa, Artificial Sequence
  Amino acid sequence of recombinant PAI-1, Q197C, G355C.
MQMSPALTCLVLGLALVFGEGSAVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISNLLGKGAVDQLTRLVLVNALYFNGCWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVAQALQKVKIEVNESCTVASSSTAVIVSARMAPEEIIMDRPFLFVVRHNPTGTVLFMGQVMEP

SEQ ID NO: 5 - DNA, 4241 bp, Artificial Sequence
  Nucleic acid sequence for hPlg-IRES2-PAI-1 expression cassette
atggaacacaaagaagtggtgttgctcctgctgctgttcctgaagtccggccagggcgagcccctggacgattacgtgaacacccagggcgccagcctgttcagcgtgaccaagaaacagctgggagccggcagcatcgaggaatgcgccgccaagtgcgaagaggacgaggaattcacctgtcgggccttccagtaccacagcaaagaacagcagtgcgtgatcatggccgagaacagaaagagcagcatcatcatcagaatgcgggacgtggtgctgttcgagaagaaggtgtacctgagcgagtgcaagaccggcaacggcaagaactaccggggcaccatgagcaagaccaagaacggcatcacctgtcagaagtggtccagcaccagcccccaccggcctagattttctccagccacccaccctagcgagggcctggaagagaactactgccggaaccccgacaacgaccctcagggcccttggtgctacaccaccgaccccgagaagagatacgactactgcgacatcctggaatgtgaagaggaatgcatgcactgcagcggcgagaactacgacggcaagatctccaagaccatgagcggcctggaatgccaggcttgggacagccagtctcctcacgcccacggctacatccccagcaagttccccaacaagaacctgaagaagaattactgcagaaaccctgaccgcgagctgcggccctggtgttttaccaccgatcctaacaagagatgggagctgtgcgatatcccccggtgcaccacacctccacctagcagcggccctacctaccagtgtctgaagggcaccggcgagaattacaggggcaacgtggccgtgaccgtgtccggccatacctgccagcattggagcgcccagaccccccacacccacaacagaacccccgagaacttcccctgcaagaatctggacgagaattattgtcgcaaccccgatggcaagagggccccctggtgtcacaccaccaacagccaggtgcgctgggagtactgcaagatccccagctgcgatagcagccccgtgtccacagaacagctggcccctacagcccctcctgagctgacacctgtggtgcaggattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactgaaagcttggtaccgagctcggatcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatgcagatgtctcccgccctgacctgcctggtgctgggcctggccctggtgttcggagagggctctgccgtgcaccacccacctagctacgtggcacacctggcctccgacttcggcgtgagggtgtttcagcaggtggcccaggccagcaaggatcgcaacgtggtgttcagcccttatggcgtggcctccgtgctggccatgctccagctgaccacaggaggagagacccagcagcagatccaggcagctatgggcttcaagatcgacgataagggaatggcacccgccctgaggcacctgtacaaggagctgatgggcccttggaataaggacgagatcagcaccacagatgccatctttgtgcagcgcgacctgaagctggtgcagggcttcatgccacacttctttcggctgttccggagcaccgtgaagcaggtggacttcagcgaggtggagagggcccgctttatcatcaacgattgggtgaagacccacacaaagggcatgatcagcaatctgctgggcaagggagcagtggatcagctgaccaggctggtgctggtgaacgccctgtacttcaatggctgctggaagaccccatttcccgacagctccacacaccggagactgttccacaagtccgatggctctacagtgagcgtgcctatgatggcccagaccaacaagttcaattatacagagtttaccacacctgacggccactactatgacatcctggagctgccataccacggcgacaccctgagcatgtttatcgccgccccttatgagaaggaggtgccactgtccgccctgacaaacatcctgtccgcccagctgatctctcactggaagggcaatatgaccaggctgccaaggctgctggtgctgcctaagttctccctggagacagaggtggacctgcggaagcctctggagaacctgggcatgaccgatatgttcagacagtttcaggccgactttacatctctgagcgatcaggagccactgcacgtggcacaggccctccagaaggtgaagatcgaggtgaacgagtcctgtaccgtggcctctagctccacagccgtgatcgtgtctgccaggatggccccagaggagatcatcatggatcggcccttcctgtttgtggtgagacacaatccaaccggcacagtgctgttcatgggccaggtcatggagccctga

SEQ ID NO: 6 - DNA, 2433 bp, Homo sapiens; identical to SEQ ID NO: 1

SEQ ID NO: 7 - protein, 810 aa, Homo sapiens; identical to SEQ ID NO: 2

SEQ ID NO: 8 - DNA, 2202 bp, Homo sapiens
atggaacacaaagaagtggtgttgctcctgctgctgttcctgaagtccggccagggcaaggtgtacctgagcgagtgcaagaccggcaacggcaagaactaccggggcaccatgagcaagaccaagaacggcatcacctgtcagaagtggtccagcaccagcccccaccggcctagattttctccagccacccaccctagcgagggcctggaagagaactactgccggaaccccgacaacgaccctcagggcccttggtgctacaccaccgaccccgagaagagatacgactactgcgacatcctggaatgtgaagaggaatgcatgcactgcagcggcgagaactacgacggcaagatctccaagaccatgagcggcctggaatgccaggcttgggacagccagtctcctcacgcccacggctacatccccagcaagttccccaacaagaacctgaagaagaattactgcagaaaccctgaccgcgagctgcggccctggtgttttaccaccgatcctaacaagagatgggagctgtgcgatatcccccggtgcaccacacctccacctagcagcggccctacctaccagtgtctgaagggcaccggcgagaattacaggggcaacgtggccgtgaccgtgtccggccatacctgccagcattggagcgcccagaccccccacacccacaacagaacccccgagaacttcccctgcaagaatctggacgagaattattgtcgcaaccccgatggcaagagggccccctggtgtcacaccaccaacagccaggtgcgctgggagtactgcaagatccccagctgcgatagcagccccgtgtccacagaacagctggcccctacagcccctcctgagctgacacctgtggtgcaggattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 9 - protein, 733 aa, Homo sapiens
MEHKEVVLLLLLFLKSGQGKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 10 - DNA, 1365 bp, Homo sapiens
atggaacacaaagaagtggtgttgctcctgctgctgttcctgaagtccggccagggcgattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 11 - protein, 454 aa, Homo sapiens
MEHKEVVLLLLLFLKSGQGDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 12 - DNA, 1056 bp, Homo sapiens
atggaacacaaagaagtggtgttgctcctgctgctgttcctgaagtccggccagggcgaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 13 - protein, 351 aa, Homo sapiens
MEHKEVVLLLLLFLKSGQGEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 14 - DNA, 807 bp, Homo sapiens
atggaacacaaagaagtggtgttgctcctgctgctgttcctgaagtccggccagggcgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 15 - protein, 268 aa, Homo sapiens
MEHKEVVLLLLLFLKSGQGAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 16 - protein, 791 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 17 - protein, 714 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 18 - protein, 435 aa, Homo sapiens
DCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 19 - protein, 332 aa, Homo sapiens
EDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 20 - protein, 249 aa, Homo sapiens
APSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 21 - DNA, 3135 bp, Artificial Sequence
  Exemplary nucleotide sequence of Plasminogen-linker-Fc fusion
atggaacacaaagaagtggtgttgctcctgctgctgttcctgaagtccggccagggcgagcccctggacgattacgtgaacacccagggcgccagcctgttcagcgtgaccaagaaacagctgggagccggcagcatcgaggaatgcgccgccaagtgcgaagaggacgaggaattcacctgtcgggccttccagtaccacagcaaagaacagcagtgcgtgatcatggccgagaacagaaagagcagcatcatcatcagaatgcgggacgtggtgctgttcgagaagaaggtgtacctgagcgagtgcaagaccggcaacggcaagaactaccggggcaccatgagcaagaccaagaacggcatcacctgtcagaagtggtccagcaccagcccccaccggcctagattttctccagccacccaccctagcgagggcctggaagagaactactgccggaaccccgacaacgaccctcagggcccttggtgctacaccaccgaccccgagaagagatacgactactgcgacatcctggaatgtgaagaggaatgcatgcactgcagcggcgagaactacgacggcaagatctccaagaccatgagcggcctggaatgccaggcttgggacagccagtctcctcacgcccacggctacatccccagcaagttccccaacaagaacctgaagaagaattactgcagaaaccctgaccgcgagctgcggccctggtgttttaccaccgatcctaacaagagatgggagctgtgcgatatcccccggtgcaccacacctccacctagcagcggccctacctaccagtgtctgaagggcaccggcgagaattacaggggcaacgtggccgtgaccgtgtccggccatacctgccagcattggagcgcccagaccccccacacccacaacagaacccccgagaacttcccctgcaagaatctggacgagaattattgtcgcaaccccgatggcaagagggccccctggtgtcacaccaccaacagccaggtgcgctgggagtactgcaagatccccagctgcgatagcagccccgtgtccacagaacagctggcccctacagcccctcctgagctgacacctgtggtgcaggattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaacggccaggccggccaagcttccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 22 - protein, 1044 aa, Artificial Sequence
  Exemplary amino acid sequence of recombinant Plasminogen-linker-Fc fusion
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNNGQAGQASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 7 aa, Artificial Sequence
  Exemplary amino acid sequence of linker (QA linker)
GQAGQAS

SEQ ID NO: 24 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 218 aa, Homo sapiens
PAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGK

SEQ ID NO: 26 - protein, 218 aa, Homo sapiens
PAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 217 aa, Homo sapiens
PAPELLGGPSVFLFPPKPKDTLMISRTPEVTCWVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 218 aa, Homo sapiens
PAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 30 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 218 aa, Artificial Sequence; identical to SEQ ID NO: 26
  Fc(lgG1-238S) (EU numbering 230-447)

SEQ ID NO: 32 - protein, 217 aa, Artificial Sequence
  Fc(lgG2-233E/234L/235L/236G) (EU numbering 230-447)
PAPELLGGPSVFLFPPKPKDTLMISRTPEVTCWVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 218 aa, Artificial Sequence
  Fc(lgG2-233E/234L/235L/236G/238S) (EU numbering 230-447)
PAPELLGGSSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 12 aa, Artificial Sequence
  Amino acid sequence of TP linker
GTPTPTPTPTGE

SEQ ID NO: 35 - protein, 15 aa, Artificial Sequence
  Exemplary GS-based linker sequence
GGGGSGGGGSGGGGS

SEQ ID NO: 36 - protein, 8 aa, Artificial Sequence
  Linker
  misc_feature (2)..(2): Xaa can be any naturally occurring amino acid
GXQAGQAS

SEQ ID NO: 37 - protein, 8 aa, Artificial Sequence
  LInker
  misc_feature (5)..(5): Xaa can be any naturally occurring amino acid
GQAGXQAS

SEQ ID NO: 38 - protein, 8 aa, Artificial Sequence
  Linker
  misc_feature (8)..(8): Xaa can be any naturally occurring amino acid
GQAGQASX

SEQ ID NO: 39 - protein, 3 aa, Artificial Sequence
  Linker
GGS

SEQ ID NO: 40 - protein, 4 aa, Artificial Sequence
  Linker
GGGS

SEQ ID NO: 41 - protein, 5 aa, Artificial Sequence
  Linker
GGGGS

(19) United States
(12) Patent Application Publication
(10) Pub. No.: US 2024/0100134 A1
(43) Pub. Date: Mar. 28, 2024

(54) FUSION PROTEINS

(71) Applicant: Monash University, Clayton (AU)

(72) Inventors: James C. Whisstock, Clayton, Victoria (AU); Ruby HP Law, Clayton, Victoria (AU); Adam J. Quek, Clayton, Victoria (AU); Christoph Hagemeyer, Clayton, Victoria (AU); Mikael Martino, Clayton, Victoria (AU); Guojie Wu, Clayton, Victoria (AU); Yen-Zhen Lu, Clayton, Victoria (AU)

(73) Assignee: Monash University, Clayton (AU)

(21) Appl. No.: 18/273,156

(22) PCT Filed: Jan. 20, 2022
(86) PCT No.: PCT/AU2022/050025

§ 371 Date: Jul. 19, 2023

(30) Foreign Application Priority Data
Jan. 20, 2021 (AU) 2021900118

(51) Int. Cl. A61K 38/48; A61P 7/02; C12N 9/68; C12N 15/62

(52) U.S. Cl. CPC A61K 38/484; A61P 7/02; C12N 9/6435; C12N 15/62; C12Y 304/21007; C07K 2319/30

(57) ABSTRACT

The present invention relates to chimeric and fusion proteins and their compositions, and the use of such proteins and compositions in the prevention and/or treatment diseases or conditions requiring plasminogen supplementation. In one aspect, the invention provides a chimeric or fusion protein comprising plasminogen and an Fc region of an antibody.

### FIELD OF THE INVENTION

The present invention relates to chimeric and fusion proteins and their compositions, and the use of such proteins and compositions in the prevention and/or treatment diseases or conditions requiring plasminogen supplementation.

### RELATED APPLICATION

This application is a national stage filing under 35 U.S.C. § 371 of international application number PCT/AU2022/050025, filed Jan. 20, 2022, which claims the benefit of Australian application number AU 2021900118, filed Jan. 20, 2021, each of which is herein incorporated by reference in its entirety.

### REFERENCE TO A SEQUENCE LISTING SUBMITTED AS A TEXT FILE VIA EFS-WEB

This application contains a Sequence Listing which has been submitted in ASCII format via EFS-Web and is hereby incorporated by reference in its entirety. Said ASCII copy, created on Jul. 19, 2023, is named M151170002US00-SEQ-KZM and is 111,608 bytes in size.

### BACKGROUND OF THE INVENTION

Plasmin is the principle fibrinolytic enzyme in mammals. This protein is a serine protease belongs to the chymotrypsin-like family that is derived from the inactive zymogen precursor plasminogen, circulating in plasma.

Plasminogen is a single-chain glycoprotein consisting of 791 amino acids with a molecular mass of approximately 92 kDa. Plasminogen is mainly synthesized in the liver and is abundant in most extracellular fluids. In plasma the concentration of plasminogen is approximately 2 μM. Plasminogen therefore constitutes a large potential source of proteolytic activity in tissues and body fluids.

Plasminogen exists in two molecular forms: Glu-plasminogen and Lys-plasminogen. The native secreted and uncleaved form has an amino-terminal (N-terminal) glutamic acid and is therefore designated Glu-plasminogen. However, in the presence of plasmin, Glu-plasminogen is cleaved at Lys76-Lys77 to become Lys-plasminogen. Compared to Glu-plasminogen, Lys-plasminogen has a higher affinity for fibrin and is activated by plasminogen activators at a higher rate, however, there is no evidence that Lys-plasminogen is found in the circulation.

Plasminogen is activated to plasmin by cleavage of the Arg561-Val562 peptide bond by either tissue-type plasminogen activator (tPA) or urokinase-type plasminogen activator (uPA). This cleavage results in an α-heavy-chain consisting of one pan-apple and five kringle domains, four of these kringle domains with lysine-binding sites and a p light-chain with the catalytic triad, namely His603, Asp646, and Ser741. The active plasmin is involved in the lysis of fibrin clots in the host. It has been shown that, upon binding to a fibrin clot, the Pan-apple domain of the native plasminogen (with an N-terminal glutamic acid Glu-plasminogen) is readily cleaved and converted into a modified Plasminogen (83 kDa) with an N-terminal lysine (Lys-plasminogen).

Two major glycoforms of plasminogen exist in human plasma: Type 1 plasminogen, which contains at least two glycosylation moieties (N-linked to N289 and O-linked to T346), and Type 2 plasminogen, which contains at least one O-linked sugar (O-linked to T346). Type 2 plasminogen is preferentially recruited to the cell surface, whereas Type 1 plasminogen is more predominantly recruited to blood clots.

Plasminogen can exist in two conformations: closed and open. The native Glu-plasminogen in the circulation is in the closed form as such that the activation site is not exposed. Once bound to the target, such as fibrin clot or cell surface receptor, via the lysine-binding sites on the kringle domains, it changes to an open conformation with its activation site exposed. The dimensions of the molecule differ significantly between these two conformations.

Plasmin is a fundamental component of the fibrinolytic system and is the main enzyme involved in the lysis of blood clots and clearance of extravasated fibrin. In addition, plasmin cleaves a wide range of biological targets including basement membrane, extracellular matrices, cell receptors, cytokines and complements. Plasminogen is therefore vital in wound healing, cell migration, tissue remodeling, angiogenesis and embryogenesis. Plasminogen has been implicated in multiple cell processes during all phases of wound healing—inflammatory, proliferative, and remodeling. These processes include fibrin degradation, platelet activation, release of cytokines and growth factors, clearance of apoptotic cells, activation of keratinocytes and epithelial-to-mesenchymal transition of fibroblasts, cell migration, and extracellular matrix degradation.

There are numerous technical difficulties associated with obtaining sufficient quantities and sufficiently pure preparations of recombinant plasminogen for use a therapeutic agent. Because of the complex structure of the full-length plasminogen molecule, bacterial expression systems have not proven useful for recombinant plasminogen production. Plasminogen is produced in the form of insoluble inclusion bodies and is not re-foldable from that state. Further, the expression of plasminogen in mammalian cells is complicated by intracellular activation of plasminogen into plasmin and the resulting cytotoxicity. Production of fully active plasminogen using insect cells is possible, however, this system is not suitable for large-scale production due to low yield.

As a consequence of the difficulty in obtaining suitable amounts and quality of recombinant plasminogen using recombinant systems, most plasminogen produced for use in clinical settings today is derived from fractionation of plasma. Obtaining plasminogen directly from human plasma presents with its own problems including the need to rely on sufficient donations of source material and risks of pathogen contamination thereof (including by viruses).

There is a need for new or improved modified plasminogen for use in treatment of conditions requiring plasminogen supplementation.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The inventors developed novel recombinant plasminogen fusion proteins comprising plasminogen linked to an Fc region of an antibody.

In one aspect, the present invention provides a chimeric or fusion protein comprising plasminogen and an Fc region of an antibody. The plasminogen may be covalently linked directly or indirectly to the Fc region of an antibody.

The plasminogen may correspond to the plasminogen sequence of any mammal. In any embodiment, the plasminogen is human plasminogen, non-human primate plasminogen, pig, mouse, rat, sheep, goat, horse, cow, cat, dog, or other mammalian plasminogen. Preferably, the plasminogen is human plasminogen.

In any embodiment of the invention, the plasminogen is selected from the group consisting of: Glu-Plg, Lys-Plg, Midi-Plg, Mini-Plg and Micro-Plg.

The plasminogen may comprise the wild-type plasminogen sequence, or may comprise a variant or modified sequence thereof. In any embodiment of the invention, the plasminogen is selected from the group consisting of: Glu-Plg, Lys-Plg, Midi-Plg, Mini-Plg and Micro-Plg. In alternative embodiments, the plasminogen may comprise a plasminogen sequence comprising amino acid substitutions at the protease active site, at the activation site, and combinations thereof and or amino acid substitutions that lead to increased protease activity.

In certain embodiments, the plasminogen comprises, consists or consists essentially of an amino acid sequence as set forth in any one of SEQ ID NOs: 2, 7, 9, 11, 13, 15, 16, 17, 18, 19 or 20, or a sequence that is at least 75%, at least 80%, at least 85%, at least 90%, at least 91%, at least 92%, at least 93%, at least 94%, at least 95%, at least 96%, at least 97%, at least 98%, at least 99% identical to the amino acid sequence as set forth in any of SEQ ID NOs: 2, 7, 9, 11, 13, 15, 16, 17, 18, 19 or 20. In one embodiment, the plasminogen comprises, consists or consists essentially of an amino acid sequence of SEQ ID NOs: 2, 7, 9, 11, 13, 15, 16, 17, 18, 19 or 20 with 0 to 8 amino acid insertions, deletions, substitutions or additions (or a combination thereof). In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof).

In one embodiment, the plasminogen does not contain a signal sequence, including any signal sequence described herein.

Preferably, the Fc region of the antibody of the chimeric or fusion protein is an Fc region of an IgG, more preferably IgG1 although the Fc region may also be derived from IgG2 or IgG3 or IgG4.

Preferably, the plasminogen polypeptide of the fusion protein is fused at the C-terminus to the Fc region. Alternatively, the plasminogen polypeptide of the fusion protein is fused via a linker at the C-terminus to the Fc region.

Preferably, the Fc region of the fusion protein comprises two immunoglobulin heavy chain fragments, more preferably the CH2 and CH3 domains of said heavy chain.

In any aspect, an Fc region of an antibody comprises, consists essentially of or consists of an amino acid sequence of any one of SEQ ID NOs: 24 to 33, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 24 to 33.

In any aspect, an Fc region of an antibody comprises, consists essentially of or consists of an amino acid sequence of any one of SEQ ID NOs: 24 to 33 having with 0 to 8 amino acid insertions, deletions, substitutions or additions (or a combination thereof). In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof).

In any aspect, the chimeric or fusion protein of the present invention includes a peptide linker between the plasminogen and the Fc region of an antibody. In one embodiment, the linker comprises or consists of amino acids. The linker may be any linker known in the art to the skilled person and may be a flexible linker (such as those comprising glycine and/or glutamine residues, or repeats of glycine and serine residues), a rigid linker (such as those comprising glutamic acid and lysine residues, flanking alanine repeats, or comprising (XP)n, wherein X is any amino acid) and/or a cleavable linker (such as sequences that are susceptible by protease cleavage).

The peptide linker may be any one or more repeats of Gly-Gly-Ser (GGS) (SEQ ID NO: 39), Gly-Gly-Gly-Ser (GGGS) (SEQ ID NO: 40) or Gly-Gly-Gly-Gly-Ser (GGGGS) (SEQ ID NO: 41) or variations thereof. In one embodiment, the linker may comprise or consist of the sequence GGGGSGGGGSGGGGS (G4S)₃. (SEQ ID N): 35)

In one embodiment, the peptide linker can include the amino acid sequence GGGGS (a linker of 6 amino acids in length) or even longer. The linker may a series of repeating glycine and serine residues (GS) of different lengths, i.e., (GS)ₙ where n is any number from 1 to 15 or more. For example, the linker may be (GS)₃ (i.e., GSGSGS) or longer (GS)₁₁ or longer. It will be appreciated that n can be any number including 1, 2, 3, 4, 5, 6, 7, 8, 9, 10, 11 or more.

The peptide linker may consist of a series of repeats of Thr-Pro (TP) comprising one or more additional amino acids N and C terminal to the repeat sequence. For example, the linker may comprise or consist of the sequence GTPTPTPTPTGE (also known as the TP5 linker), SEQ ID NO: 34.

In a preferred embodiment, the linker comprises or consists of the amino acid sequence in SEQ ID NO: 23 (GQAGQAS, which may also be referred to as a “QA” linker), or a sequence having at least 90% identity thereto. Variations of the “QA” linker include: GXQAGQAS (SEQ ID NO: 35); GQAGXQAS (SEQ ID NO: 37), GQAGQASX (SEQ ID NO: 38), wherein X is any amino acid. In certain embodiments, X is a lysine residue and/or the linker may further include one or more lysine residues.

In any aspect, the chimeric or fusion protein comprises or consists of the amino acid sequence as set forth in SEQ ID NO: 22.

In preferred aspects, the linker is a flexible linker. In preferred aspects, the linker is not a rigid linker. In further preferred aspects, the linker is a cleavable linker, and is susceptible to cleavage upon activation of the plasminogen portion of the fusion protein. Examples of various flexible and rigid linkers are known to the skilled person and are described for example, in Chen et al., (2013) Advanced Drug Delivery Reviews, 65: 1357-1369.

In any aspect, the linker does not comprise the sequence GTPTPTPTPTGE (SEQ ID NO: 34).

In another aspect, the present invention includes a nucleic acid comprising or consisting of a nucleotide sequence encoding a chimeric or fusion protein of the invention.

In any aspect, a nucleic acid of the invention comprises a nucleotide sequence that encodes any plasminogen as described herein. Preferably, the nucleotide sequence that encodes a plasminogen comprises, consists or consists essentially of SEQ ID NOs: 1, 5, 6, 8, 10, 12 or 14, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% sequence identity to the sequence set forth in any one of SEQ ID NOs: 1, 5, 6, 8, 10, 12 or 14.

In any aspect, a nucleic acid of the invention comprises a nucleotide sequence that encodes a linker between a nucleotide sequence encoding plasminogen and a nucleotide sequence encoding an Fc region of an antibody. The linker may be any one described herein. In one embodiment, the nucleotide sequence encodes a linker that comprises or consist of SEQ ID NO: 23.

In any aspect, a nucleic acid of the invention comprises a nucleotide sequence that encodes any Fc region of an antibody as described herein. Preferably, the nucleotide sequence that encodes an Fc region of an antibody comprises, consists or consists essentially of any one of SEQ ID NOs: 24 to 33, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% sequence identity to the sequence set forth in any one of SEQ ID NOs: 24 to 33.

In another aspect, the present invention also provides a vector or construct comprising a nucleic acid of the invention. Preferably, the vector or construct comprises a further nucleotide sequence encoding PAI-1 or variant thereof. Preferably, the nucleotide sequence encoding a chimeric or fusion protein of the invention and nucleotide sequence encoding PAI-1 or variant thereof are operably linked to a promoter for enabling the expression of the polynucleotides. In certain embodiments, the chimeric or fusion protein of the invention and PAI-1 are encoded in a single polynucleotide construct to enable bicistronic expression. In certain embodiments, the vector or construct comprises an internal ribosome entry site (IRES) between the nucleotide sequence encoding a chimeric or fusion protein of the invention and nucleotide sequence encoding PAI-1 or variant thereof that allows for translation initiation in a cap-independent manner.

In another aspect, the present invention provides a host cell comprising a vector or construct of the invention as described herein.

The host cell is preferably a mammalian host cell, including but not limited to a cell selected from the group consisting of: Expi293, variants of Expi293, CHO (Chinese Hamster Ovary) cells and derivatives thereof, HeLa (Human cervical cancer) cells, COS and Vero cells.

In certain embodiments, the nucleotide sequence encoding a PAI-1 or variant thereof comprises, consists or consists essentially of the nucleic acid sequence of SEQ ID NO: 3, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% identity to the sequence set forth in SEQ ID NO:3.

In certain embodiments, the plasminogen encoded a nucleotide sequence in a nucleic acid, vector or construct of the invention comprises, consists or consists essentially of, or has an amino acid sequence that is at least 75%, at least 80%, at least 85%, at least 90%, at least 91%, at least 92%, at least 93%, at least 94%, at least 95%, at least 96%, at least 97%, at least 98%, at least 99% identical to the amino acid sequence set forth in any one of SEQ ID NOs: 2, 7, 9, 11, 13, 15, 16, 17, 18, 19 or 20.

In one embodiment, the PAI-1 comprises, consists, or consists essentially of the amino acid sequence as shown in SEQ ID NO: 4. Alternatively, the PAI-1 sequence may comprise, consist or consist essentially of the sequence for unmodified (i.e., wild-type) PAI-1, wherein the wild-type sequence consists of the sequence of SEQ ID NO: 4, wherein the residues at positions 197 and 355 are glutamine and glycine, respectively.

In one embodiment, the vector or construct comprises a nucleic acid sequence as shown in SEQ ID NO: 5, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% sequence identity to the sequence set forth in SEQ ID: 5.

In another aspect, the present invention provides method for producing a chimeric or fusion protein of the invention, the method comprising:
(i) providing a host cell comprising a first nucleic acid encoding a chimeric or fusion protein of the invention and a second nucleic acid encoding a plasminogen activator inhibitor;
(ii) culturing said host cell in a suitable culture medium under conditions to effect expression of the chimeric or fusion protein from the first nucleic acid and plasminogen activator inhibitor from the second nucleic acid.

Preferably, the present invention provides a method for producing plasminogen, the method comprising:
(i) providing a host cell comprising a first nucleic acid encoding a chimeric or fusion protein of the invention and a second nucleic acid encoding plasminogen activator inhibitor-1 (PAI-1) or variant thereof;
(ii) culturing said host cell in a suitable culture medium under conditions to effect expression of the chimeric or fusion protein from the first nucleic acid and PAI-1 or variant thereof from the second nucleic acid.

In one aspect, the present invention provides a method of producing a chimeric or fusion protein of the invention, the method comprising the steps of:
(a) providing a first nucleic acid encoding a chimeric or fusion protein of the invention,
(b) providing a second nucleic acid encoding PAI-1 or variant thereof;
wherein the first and the second nucleic acids are operably linked to a promoter for enabling the expression of the nucleic acids encoding the chimeric or fusion protein of the invention and PAI-1 or variant thereof,
(c) providing a host cell,
(d) transforming or transfecting the host cell with the nucleic acids of a) and b)
(e) providing cell culture media,
(f) culturing the transformed or transfected host cell in the cell culture media under conditions sufficient for expression of the nucleic acids encoding the chimeric or fusion protein of the invention and the PAI-1 or variant thereof, and
optionally (g) recovering or purifying the chimeric or fusion protein of the invention from the host cell and/or the cell culture media.

Preferably, the plasminogen activator inhibitor is PAI-1. More preferably, the PAI-1 comprises, consists, or consists essentially of the amino acid sequence as shown in SEQ ID NO: 4. Alternatively, the PAI-1 sequence may comprise, consist or consist essentially of the sequence for unmodified (i.e., wild-type) PAI-1, wherein the wild-type sequence consists of the sequence of SEQ ID NO: 4, wherein the residues at positions 197 and 355 are glutamine and glycine, respectively.

In another aspect, the present invention provides isolated, purified, substantially purified or recombinant chimeric or fusion protein produced by a method of the invention as. The plasminogen included in the chimeric or fusion protein may be any one described herein, for example may comprises, consists or consists essentially of an amino acid sequence as set forth in any one of SEQ ID NOs: 2, 7, 9, 11, 13, 15, 16, 17, 18, 19 or 20, or a sequence that is at least 75%, at least 80%, at least 85%, at least 90%, at least 91%, at least 92%, at least 93%, at least 94%, at least 95%, at least 96%, at least 97%, at least 98%, at least 99% identical to the amino acid sequence as set forth in any of SEQ ID NOs: 2, 7, 9, 11, 13, 15, 16, 17, 18, 19 or 20. Preferably, the plasminogen does not contain a signal sequence, including any signal sequence described herein.

In another aspect, there is provided a dimeric protein formed from covalently bonded monomers of the chimeric or fusion protein described herein. Preferably dimerisation occurs via cysteine residues present in the Fc portion of the chimeric or fusion protein.

In another aspect, the present invention provides a composition comprising a chimeric or fusion protein of the invention and plasminogen activator inhibitor, preferably PAI-1 or variant thereof, isolated, purified or substantially purified from the culture media from a method of the invention as described herein.

In a further aspect, the present invention provides isolated, purified, substantially purified, or recombinant plasmin derived or obtained from plasminogen that is produced by a method of the invention as described herein. As such, the present invention provides a chimeric or fusion protein comprising plasmin and an Fc region of an antibody. The plasmin may be covalently linked directly or indirectly to the Fc region of an antibody.

Still further, the present invention provides for the use of isolated, purified, substantially purified or recombinant chimeric or fusion protein (or chimeric or fusion protein comprising plasmin derived therefrom) in a method of treating a condition in an individual, wherein the condition requires administration of exogenous plasminogen (or plasmin).

In another aspect, the present invention provides a composition comprising a chimeric or fusion protein of the invention, nucleic acid of the invention, a vector or expression construct of the invention or a host cell of the invention, and a pharmaceutically or physiologically acceptable carrier, diluent or excipient.

In another aspect, the present invention provides, a method of inducing or promoting lysis of a pathological fibrin deposit in a subject, comprising administering a chimeric or fusion protein, nucleic acid, vector or expression construct, or host cell of the invention to the subject, thereby inducing or promoting lysis of a pathological fibrin deposit in the subject.

In another aspect, the present invention provides, use of a chimeric or fusion protein, nucleic acid, vector or expression construct, or host cell of the invention in the manufacture of a medicament for inducing or promoting lysis of a pathological fibrin deposit in a subject.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Size exclusion chromatography of rPlg-Fc. (A) Coomassie stained 10% SDS-PAGE of rPlg-Fc. Protein bands are observed at about 150 kDa (monomer) under reducing conditions and at about 300 kDa (dimer) under non-reducing conditions. Expected size of the monomeric fusion protein without glycosylation: 114.6 kDa. (B) Elution profile from a Superdex 200 10/30 column, showing rPlg-Fc is purified as a single species.

FIG. 2: Progress curve showing tPA and uPA-mediated activation of rPlg and rPlg-Fc fusion. Activation of rPlg and rPlg-Fc was measured by the hydrolysis of plasmin fluorogenic substrate H-Ala-Phe-Lys-AMC. rPlg-Fc alone does not show any hydrolytic activity, as expected. In the presence of tPA or uPA, comparable hydrolytic activity for rPlg and rPlg-Fc is observed.

FIG. 3: Michelis-Menten analysis of rPlg and rPlg-Fc activation by tPA and uPA in the presence of 10 mM EACA. Results from activation of rPlg and rPlg-Fc by tPA (A) and uPA (B) are shown. The result indicates that the kinetics of rPlg and rPlg-Fc activation are similar (as indicated by the K_{M} and Vₘₐₓ).

FIG. 4: Kinetics of Inhibition by alpha2-antiplasmin (AP). Progress curve in the presence of plasmin fluorogenic substrate H-Ala-Phe-Lys-AMC, shows a dose dependent inhibition of plasmin activity generated by tPA from (A) rPlg and (B) rPlg-Fc. (C) The normalised plasmin activity is plotted against an increasing molar ratio of AP to Plg. The result shows that plasmin generated from rPlg-Fc and rPlg is inhibited by AP. (D) The concentration of AP required to inhibit 50% of plasmin activity (/C₅₀) is derived from (C), and is comparable for both rPlg and rPlg-Fc.

FIG. 5: Stability of rPlg-Fc at different temperatures. rPlg-Fc was stored at 37° C., room temperature, 4° C. and −20° C. as indicated for up to 7 days (D0-D7) and analysed by Coomassie stained 10% SDS-PAGE under reducing conditions. No degradation product is detectable amongst all the samples analysed.

FIG. 6: Stability of rPlg-Fc in human plasma. Fluorescently labelled native Plg (A) rPlg (B), rPlg-Fc (C), rPlg-Fc with QA linker (D), rPlg-Fc with TP linker (E) or rPlg-Fc with (G4S)₃ linker (F) were mixed with human plasma from blood bank at 2 mM (a physiological concentration) and stored at 37° C. for up to ˜10 days as indicated. As a control, the samples were mixed with HBS (not shown). Integrity of proteins was analysed by fluorescence scanning of 10% SDS-PAGE under reducing and non-reducing conditions. No degradation was detectable in any of the samples following two days of incubation. When the incubation period was longer than 2 days, some degradation was observed for the native Plg sample, while total hydrolysis was observed for the rPlg. No degradation was observed for the rPlg-Fc suggesting it is the most stable form in plasma. No breakdown was observed in any of the HBS samples (not shown).

FIG. 7: In vivo stability of rPlg and rPlg-Fc in mice. Fluorescently labelled rPlg or unlabelled rPlg-Fc were injected intravenously at 25 mg/kg; two mice were used per timepoint. (A) The stability of rPlg in plasma was monitored via fluorescence signal following injection. The plasma half-life was estimated to be ˜5 hours. (B) The stability of intact rPlg-Fc was determined via a sandwich ELISA assay in which an anti-Plg-specific monoclonal antibody was used as the capture antibody and an anti-Fc antibody as the reporter antibody. The plasma half-life was estimated to be ˜27 hours.

FIG. 8: Upon activation, the Fc portion dissociates from Plg/Plm. rPlg-Fc was activated by tPA and uPA for up to 120 min as indicated. The sample was analysed by 12% SDS-PAGE followed by Coomassie staining. The results show that Plg-Fc is rapidly cleaved at the linker region generating Plg and Fc fragments (20 min); the activation by plasmin activators cleaves Plg into the two-chained Plm consisting of a heavy chain and a light chain, (20-120 min). uPA is more active as a Plg activator: all full-length Plg-Fc is cleaved to Plm and Fc after 40 min; ˜80% of Plg is cleaved to Plm after 120 min. For tPA: all full-length Plg-Fc is cleaved to Plg and Fc after 90 min ˜50% of Plg is activated after 120 min.

FIG. 9: Time-course of Plg activation by tPA and uPA. A. rPlg and rPlg-Fc were activated by tPA and uPA for up to 120 min as indicated. Sample was analysed by Plm activity using H-Ala-Phe-Lys-AMC (Plm fluorogenic substrate), the progression curves are shown and samples are as labelled. B. Enzyme activity of rPlg-Fc proteins comprising 3 different linkers was assessed (QA, TP and GS linkers). Following activation with either tPA or uPA, at the 40 minute mark, 4 μL sample was mixed with 5 μL of 2 mM AKF-AMC substrate, 91 μL of assay buffer. Enzyme activity was measured at 37° C. and as ΔFU/min. Enzyme activity was comparable for all three proteins.

FIG. 10: Synthetic clot lysis by rPlg and rPlg-Fc. (A) progression curves of synthetic clot lysis were recorded for both rPlg and rPlg-Fc. Fibrinolysis was initiated with addition of 45 nM Plg and 10 nM tPA. (B) The time required to achieve full lysis is derived from (A), and it is comparable for rPlg and rPlg-Fc.

FIG. 11: Chronic wound healing by rPlg and rPlg-Fc. Promotion of wound healing as assessed by percentage of wound closure in a diabetic mouse model following administration of PBS, rPlg RASA (inactive), Fc only, rPlg (wild-type; WT) and rPlg-Fc (WT). rPlg-Fc promotes significantly greater wound closure compared to rPlg.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The general chemical terms used in the formulae herein have their usual meaning.

### Plasminogen

Plasminogen is the inactive precursor form of plasmin, the principal fibrinolytic enzyme in mammals. Plasmin also plays an important role in cell migration, tissue remodeling, and bacterial invasion. Plasmin is a seine protease that preferentially cleaves Lys-Xaa and Arg-Xaa bonds with higher selectivity than trypsin. Plasminogen activators such as tissue plasminogen activator (tPA) or urokinase cleave human plasminogen molecule at the Arg₅₆₀-Val₅₆₁ bond to produce active plasmin. The two resulting chains of plasmin are held together by two interchain disulphide bridges. The light chain (25 kDa) carries the catalytic center (which comprises the catalytic triad) and shares sequence similarity with trypsin and other serine proteases. The heavy chain (60 kDa) consists of five highly similar triple-loop structures called kringles. Some of the kringles contain lysine binding sites that mediates the plasminogen/plasmin interaction with fibrin. Plasmin belongs to peptidase family Si.

The amino acid sequence of human Glu-Plg is provided in SEQ ID NO: 2 (see also SEQ ID NO:6). SEQ ID NO:16 shows the “mature” amino acid sequence, i.e. after cleavage of the signal peptide.

It will be understood that the present invention includes the recombinant production of plasminogen from human and non-human sources. Accordingly, the plasminogen produced according to the present methods may comprise of consist of the amino acid sequence any mammalian plasminogen or plasminogen variant. In any embodiment, the plasminogen is human plasminogen, non-human primate plasminogen, pig, mouse, rat, hamster, sheep, goat, horse, cow, cat, dog, or other mammalian plasminogen. Preferably, the plasminogen is human plasminogen.

Furthermore, the invention includes expression of functional variants of plasminogen including but not limited to those further described herein. More specifically, the present invention contemplates methods for the recombinant production of Glu-plasminogen (Glu-Plg), Lys-plasminogen (Lys-Plg), and mini-, midi- and micro-plasminogens.

Lys-plasminogen is an N-truncated form of Glu-Plg that is formed from the cleavage of Glu-plasminogen by plasmin. Lys-plasminogen exhibits higher affinity for fibrin compared to Glu-Plg and is better activated by uPA and tPA.

The amino acid sequence of human Lys-plasminogen is provided in SEQ ID NO: 9. SEQ ID NO:17 shows the “mature” amino acid sequence, i.e., after cleavage of the signal peptide.

Midi-plasminogen comprises kringle domains 4 and 5 and the light chain (serine protease domain) of plasminogen. It is formed by cleavage of kringle domains 1 to 3 from Glu-plasminogen.

The amino acid sequence of human midi-plasminogen is provided in SEQ ID NO:11. SEQ ID NO:18 shows the “mature” amino acid sequence, i.e., after cleavage of the signal peptide.

Mini-plasminogen (also known as 442Val-Plg or neoplasminogen) results from the action of elastase on Glu-plasminogen at residue 442 (located within Kringle domain 4). Thus mini-plasminogen comprises part of kringle domain 4, kringle domain 5 and the serine protease domain of plasminogen. The amino acid sequence of human mini-plasminogen is provided in SEQ ID NO:13. SEQ ID NO:19 shows the “mature” amino acid sequence, i.e., after cleavage of the signal peptide.

Micro-plasminogen consists of the proenzyme domain of plasminogen with a stretch of connecting peptide and a few residues of kringle 5 attached at its N-terminal end. It is produced by the action of plasmin on plasminogen. Thus, micro-plasminogen (or micro-Plg) comprises the light chain of plasminogen (serine protease domain) and no kringle domains. (See, for example, Shi et al. (1980) J Biol. Chem. 263:17071-5). Like plasminogen, microplasminogen is activated by tPA and urokinase to form a proteolytically active molecule. Human microplasmin has a molecular weight of approximately 29 kDa and has a lower affinity for fibrin when compared with plasmin.

The amino acid sequence of human micro-plasminogen is provided in SEQ ID NO:15. SEQ ID NO:20 shows the “mature” amino acid sequence, i.e., after cleavage of the signal peptide.

Other variants: eg: variants of plasminogen which comprise modifications or mutations in the lysine binding sites found in the kringle domains. It will be appreciated that the methods of the invention lend themselves to expression of any of a number of Plasminogen variants, including but not limited to recombinant plasminogen having a modification at one or more sites.

### Fc Region of an Antibody

The term “Fc region” herein is used to define a C-terminal region of an immunoglobulin heavy chain that contains at least a portion of the constant region. In other words, the Fc region contains two heavy chain fragments comprising the CH2 and CH3 domains of an antibody. In the context of the present invention, the Fc region comprises two heavy chain fragments, preferably the CH2 and CH3 domains of said heavy chain. The two heavy chain fragments are held together by two or more disulfide bonds and by hydrophobic interactions of the CH3 domains. The heavy chain constant domains that correspond to the different classes of immunoglobulins are called α, δ, ε, γ, and μ, respectively.

In some aspects, the fusion protein does not exhibit any effector function or any detectable effector function. “Effector functions” or “effector activities” refer to those biological activities attributable to the Fc region of an antibody, which vary with the antibody isotype. Examples of antibody effector functions include: C1q binding and complement dependent cytotoxicity (CDC); Fc receptor binding; antibody dependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006); WO 2013/120929 Al).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

In some aspects, the Fc region includes mutations to the complement (C1q) and/or to Fc gamma receptor (FcγR) binding sites. In some aspects, such mutations can render the fusion protein incapable of antibody directed cytotoxicity (ADCC) and complement directed cytotoxicity (CDC).

The Fc region as used in the context of the present invention does not trigger cytotoxicity such as antibody-dependent cellular cytotoxicity (ADCC) or complement dependent cytotoxicity (CDC).

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an anti-inflammatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

### Linkers

Moreover, the herein provided fusion proteins may comprise a linker (or “spacer”). In the context of the present invention, the polypeptide comprising or consisting of the amino acid sequence of plasminogen is fused via a linker at the C-terminus to the Fc region or Fc receptor binding domain.

A linker is usually a peptide having a length of up to 20 amino acids. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids. For example, the herein provided fusion protein may comprise a linker between the polypeptide comprising or consisting of an amino acid sequence of a plasminogen (or plasminogen derivative or related polypeptide as described herein, and the Fc region of the antibody, such as between the N-terminus of the Fc regions/FcR binding domains and the C-terminus of the polypeptide. Such linkers have the advantage that they can make it more likely that the different polypeptides of the fusion protein fold independently and behave as expected.

Thus, in the context of the present invention the polypeptide comprising or consisting of an amino acid sequence of plasminogen and the Fc region of an antibody or Fc receptor binding domain may be comprised in a single-chain multi-functional polypeptide.

In some aspects, the fusion protein of the present invention includes a peptide linker. The skilled person will be familiar with the design and use of various peptide linkers comprised of various amino acids, and of various lengths, which would be suitable for use as linkers in accordance with the present invention. The linker may comprise various combinations of repeated amino acid sequences.

The linker may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage). Examples of such linkers are known to the skilled person and are described for example, in Chen et al., (2013) Advanced Drug Delivery Reviews, 65:1357-1369.

In some aspects, the peptide linker may include the amino acids glycine and serine in various lengths and combinations. In some aspects, the peptide linker can include the sequence Gly-Gly-Ser (GGS), Gly-Gly-Gly-Ser (GGGS) or Gly-Gly-Gly-Gly-Ser (GGGGS) and variations or repeats thereof. In some aspects, the peptide linker can include the amino acid sequence GGGGS (a linker of 6 amino acids in length) or even longer. The linker may a series of repeating glycine and serine residues (GS) of different lengths, i.e., (GS)ₙ where n is any number from 1 to 15 or more. For example, the linker may be (GS)₃ (i.e., GSGSGS) or longer (GS)₁₁ or longer. It will be appreciated that n can be any number including 1, 2, 3, 4, 5, 6, 7, 8, 9, 10, 11 or more. Fusion proteins having linkers of such length are included within the scope of the present invention. Similarly, the linker may be a series of repeating glycine residues separated by serine residues. For example (GGGGS)₃ (i.e., the linker may comprise the amino acid sequence GGGGSGGGGSGGGGS (G4S)₃) and variations thereof.

The peptide linker may consist of a series of repeats of Thr-Pro (TP) comprising one or more additional amino acids N and C terminal to the repeat sequence. For example, the linker may comprise or consist of the sequence GTPTPTPTPTGE (SEQ ID NO: 34) (also known as the TP5 linker).

In a preferred embodiment, the linker comprises or consists of the amino acid sequence in SEQ ID NO: 23 which comprises a repeat of the sequence GQA, followed by a serine (i.e., GQAGQAS). The invention also contemplates the use of variations to the GQA linker sequence, including for example, the inclusion of additional residues such as lysine, at various positions throughout the linker. In certain embodiments, the linker may comprise or consist of the amino acid sequence: GXQAGQAS (SEQ ID NO: 36), GQAGXQAS (SEQ ID NO: 37), or GQAGQASX (SEQ ID NO: 38), where X is any amino acid.

In further aspects, the linker may be a short and/or alpha-helical rigid linker (e.g. A(EAAAK)3A, PAPAP or a dipeptide such as LE).

In certain aspects, the linker may be flexible and cleavable. Such linkers preferably comprise one or more recognition sites for a protease to enable cleavage.

### PAI-1

Plasminogen activator inhibitor-1 (PAI-1) also known as endothelial plasminogen activator inhibitor or serpin E1 is a protein that in humans is encoded by the SERPINE1 gene. PAI-1 is a serine protease inhibitor (serpin) that functions as the principal inhibitor of tissue plasminogen activator (tPA) and urokinase (uPA), the activators of plasminogen. In vivo, PAI-1 is thus one or the key inhibitors of fibrinolysis.

Other plasminogen activator inhibitors include plasminogen activator inhibitor-2 (PAI-2), protein C inhibitor (PAI-3) and the protease nexin-1 (SERPINE2), which acts as an inhibitor of tPA and urokinase. The present inventors have found however, that the methods of the invention have particular utility when PAI-1 or a variant thereof, is co-expressed with plasminogen.

The amino acid sequence of human PAI-1, wherein the sequence is modified at Q197 and G355 to introduce cysteine residues, is provided in SEQ ID NO: 4.

Exemplary nucleic acid and amino acid sequences for PAI-2 and PAI-3 are provided in NCBI accession numbers NM_002575.3 and NM_000624.6, respectively.

It will be understood that the present invention also contemplates the use of “wild-type” PAI-1 (i.e., wherein the sequence is not modified at residues 197 or G355 as shown in SEQ ID NO: 4).

As used herein, the term “mutant” with respect to a mutant polypeptide or mutant polynucleotide is used interchangeably with “variant.” A variant with respect to a given reference sequence can include naturally occurring allelic variants. A “variant” includes any protein or amino acid sequence comprising at least one amino acid mutation with respect to wild-type. Mutations may include substitutions, insertions, and deletions. Preferably the variant retains the capacity to inhibit a plasminogen activator to the same exact as wildtype, or to a level of at least about 99%, 98%, 97%, 96%, 96%, 04%, 93%, 92%, 91%, 90%, 85%, or 80% of wildtype. For example a PAI-1 variant retains the capacity to inhibit a plasminogen activator to a level of at least about 99%, 98%, 97%, 96%, 96%, 04%, 93%, 92%, 91%, 90%, 85%, or 80% of wildtype PAI-1. The wildtype PAI-1 may be any described herein including SEQ ID NO: 4. In one embodiment, a variant of PAI-1 is not PAI-2 or PAI-3. Preferably, a PAI-1 variant has greater potency at inhibiting tPA and/or uPA compared to PAI-2, PAI-3, or PAI-2 and PAI-3.

### Nucleic Acids

An “isolated” nucleic acid molecule is a nucleic acid molecule that is identified and separated from at least one contaminant nucleic acid molecule with which it is ordinarily associated in the natural source. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in cells that ordinarily express, for example, plasminogen, where, for example, the nucleic acid molecule is in a chromosomal location different from that of natural cells.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides of the invention can be synthesised according to methods well known in the art, as described by way of example in Sambrook et al (1989, Molecular Cloning—a laboratory manual; Cold Spring Harbor Press).

The polynucleotide molecules of the present invention may be provided in the form of an expression cassette which includes control sequences operably linked to the inserted sequence, thus allowing for expression of the polypeptide of the invention in vivo in a targeted subject. These expression cassettes, in turn, are typically provided within vectors (e.g., plasmids or recombinant viral vectors) which are suitable for use as reagents for nucleic acid immunization. Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences.

Furthermore, it will be appreciated that the compositions and products of the invention may comprise a mixture of polypeptides and polynucleotides. Accordingly, the invention provides a composition or product as defined herein, wherein in place of any one of the polypeptide is a polynucleotide capable of expressing said polypeptide.

Expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art.

Thus, the methods of the present invention include delivering such a vector to a cell and allowing transcription from the vector to occur. Preferably, a polynucleotide of the invention or for use in the invention in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extra-chromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus. Thus, a peptide of the invention may be provided to an individual by expression from cells within the individual, and secretion from those cells.

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6: 47 (1988)), and established cell lines such as the COS-7, C127, 3T3, CHO, HeLa, and BHK cell lines, and the like. The skilled person is aware that the choice of expression host has ramifications for the type of polypeptide produced. For instance, the glycosylation of polypeptides produced in yeast or mammalian cells (e.g., COS-7 cells) will differ from that of polypeptides produced in bacterial cells, such as Escherichia coli.

### Polypeptides

“Isolated,” when used to describe the various polypeptides disclosed herein, means the polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes polypeptide in situ within recombinant cells, since at least one component of the polypeptide natural environment will not be present. Ordinarily, however, isolated polypeptide will be prepared by at least one purification step.

A “fragment” is a portion of a polypeptide of the present invention that retains substantially similar functional activity or substantially the same biological function or activity as the polypeptide, which can be determined using assays described herein.

“Percent (%) amino acid sequence identity” or “percent (%) identical” with respect to a polypeptide sequence, i.e. a polypeptide of the invention defined herein, is defined as the percentage of amino acid residues in a candidate sequence that are identical with the amino acid residues in the specific polypeptide of the invention, after aligning the sequences and introducing gaps, if necessary, to achieve the maximum percent sequence identity, and not considering any conservative substitutions as part of the sequence identity.

Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, CA). After alignment of amino acid sequences with ClustalW, the percent amino acid identity can be assessed. A non-limiting examples of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, CA, USA). When utilizing the ALIGN program for comparing amino acid sequences, a PAM 120 weight residue table, a gap length penalty of 12, and a gap penalty of 4 can be used.

The polypeptide desirably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo.

The polypeptide can be prepared by any of a number of conventional techniques. The polypeptide can be isolated or purified from a naturally occurring source or from a recombinant source. Recombinant production is preferred. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1982); Sambrook et al., Molecular Cloning A Laboratory Manual, 2nd ed. (Cold Spring Harbor Laboratory, 1989). The fragment can be transcribed and the polypeptide subsequently translated in vitro. Commercially available kits also can be employed (e.g., such as manufactured by Clontech, Palo Alto, Calif.; Amersham Pharmacia Biotech Inc., Piscataway, N.J.; InVitrogen, Carlsbad, Calif., and the like). The polymerase chain reaction optionally can be employed in the manipulation of nucleic acids.

The term “conservative substitution” as used herein, refers to the replacement of an amino acid present in the native sequence in the peptide with a naturally or non-naturally occurring amino acid or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

Conservative amino acid substitution tables providing functionally similar amino acids are well known to one of ordinary skill in the art. The following six groups are examples of amino acids that may be considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

As naturally occurring amino acids are typically grouped according to their properties, conservative substitutions by naturally occurring amino acids can be determined bearing in mind the fact that replacement of charged amino acids by sterically similar non-charged amino acids are considered as conservative substitutions. For producing conservative substitutions by non-naturally occurring amino acids it is also possible to use amino acid analogs (synthetic amino acids) well known in the art. A peptidomimetic of the naturally occurring amino acid is well documented in the literature known to the skilled person and non-natural or unnatural amino acids are described further below. When affecting conservative substitutions the substituting amino acid should have the same or a similar functional group in the side chain as the original amino acid.

The phrase “non-conservative substitution” or a “non-conservative residue” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH2)5-COOH]—CO— for aspartic acid. Non-conservative substitution includes any mutation that is not considered conservative.

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Alterations of the native amino acid sequence to produce mutant polypeptides, such as by insertion, deletion and/or substitution, can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used, such as disclosed in Walder et al., Gene 42: 133 (1986); Bauer et al., Gene 37: 73 (1985); Craik, Biotechniques, 12-19 (January 1995); and U.S. Pat. Nos. 4,518,584 and 4,737,462. A preferred means for introducing mutations is the QuikChange Site-Directed Mutagenesis Kit (Stratagene, LaJolla, Calif.).

The terms “N-terminal” and “C-terminal” are used herein to designate the relative position of any amino acid sequence or polypeptide domain or structure to which they are applied. The relative positioning will be apparent from the context. That is, an “N-terminal” feature will be located at least closer to the N-terminus of the polypeptide molecule than another feature discussed in the same context (the other feature possible referred to as “C-terminal” to the first feature). Similarly, the terms “5′-” and “3′-” can be used herein to designate relative positions of features of polynucleotides.

A recombinant polypeptide made in accordance with the methods of the present invention may also be modified by, conjugated or fused to another moiety to facilitate purification of the polypeptides, or for use in immunoassays using methods known in the art. For example, a polypeptide of the invention may be modified by glycosylation, acetylation, phosphorylation, amidation, derivatization by known protecting/blocking groups, proteolytic cleavage, etc.

Modifications contemplated herein include, but are not limited to, modification to side chains, incorporating of unnatural amino acids and/or their derivatives during polypeptide synthesis and the use of crosslinkers and other methods which impose conformational constraints on the polypeptides of the invention. Any modification, including post-translational modification, that reduces the capacity of the molecule to form a dimer is contemplated herein. An example includes modification incorporated by click chemistry as known in the art. Exemplary modifications include glycosylation.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-y-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The polypeptides referred to herein as having an N-terminal domain “homologous to a kringle domain of native human plasminogen” exhibit structural and functional characteristics similar to native kringle domains of plasminogen. Further, the polypeptides referred to herein as having an N-terminal domain “homologous to kringle 1” exhibit characteristics similar to native kringle 1, at least to the extent that the polypeptides can have a higher affinity for o-aminocarboxylic acids (and functional homologs such as trans-4-aminomethylcyclohexane-15 carboxylic acid, a cyclic acid) than kringle 5. See, e.g., Chang, Y., et al., Biochemistry 37:3258-3271 (1998), incorporated herein by reference, for conditions and protocols for comparison of binding of isolated kringle domain polypeptides to aminopentanoic acid (5-APnA); 6-aminohexanoic acid (6-AHxA), also known as epsilon-aminocaprioic acid (EACA); 7-aminoheptanoic acid (7-AHpA); and trans-4aminomethylcyclohexane-1-carboxylic acid (t-AMCHA). References to kringle domains “homologous to kringle 4” are defined similarly, as noted above regarding the phrase “homologous to kringle 1.” That is, they exhibit functional characteristics similar to kringle 4 of native human plasminogen as discussed above. These polypeptides also bind immobilized lysine as described above.

The polypeptides made according to the methods of the present invention bind immobilized lysine. As used herein, the phrase “binding immobilized lysine” means that the polypeptides so characterized are retarded in their progress relative to proteins that do not bind lysine, when subjected to column chromatography using lysine-SEPHAROSE as the chromatographic media. Typically, the polypeptides of the invention can be eluted from such chromatographic media (lysine affinity resins) using solutions containing the specific ligand, e.g., EACA, as eluants.

### Cell Culture

Persons skilled in the art will be familiar with standard methods for transfecting host cells, such as mammalian cells, with a nucleic acid vector and culturing the host cell in suitable conditions for expressing genes encoded by the vector. Representative methods for transfection and culturing of mammalian cells to produce recombinant protein are described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989).

Means for introducing the isolated nucleic acid, vector or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used in accordance with the present invention may be cultured in a variety of media, depending on the cell type used. Commercially available media such as Ham's FI0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

Moreover, the skilled person will be familiar with methods for purifying expressed recombinant protein from cell culture media, including using size exclusion and affinity chromatography methods, and combinations thereof.

Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., lysine affinity column), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Assaying the Activity of the Recombinant Plasminogen

The recombinant plasminogen fusion protein produced according to the present invention (or the recombinant plasmin derived therefrom) can be assessed for biological activity using standard methods known in the art and as described later herein in the Examples.

For example, the recombinant plasminogen fusion protein can be converted to plasmin via cleavage with tPA or uPA using standard techniques. Cleavage of recombinant plasminogen with tPA yields heavy and light chains comprising residues Glu₁-Arg₅₆₁ and Val₅₆₂-Asn₇₉₁, respectively (for an example of the method for converting plasminogen to plasmin, see Mutch and Booth, Chapter 20 in Hemostasis and Thrombosis: Basic Principles and Clinical Practice by Victor J. Marder, William C. Aird, Joel S. Bennett, Sam Schulman, and II Gilbert C. White, incorporated herein by reference).

Further, the ability of the recombinant plasminogen to bind to physiological binding targets or ligands can be assessed using conventional techniques. For example, binding to recombinant plasminogen (or plasmin derived therefrom) can be assessed in relation to binding to alpha 2-antiplasmin (α2-AP) and streptokinase. (Methods for assessing binding to α2-AP and to streptokinase are described in, for example, Horvath et al., (2011) Methods in Enzymology, 501: 223-235 and in Zhang et al., (2012) Journal of Biological Chemistry, 287: 42093-42103, respectively, the contents of which are hereby incorporated by reference.

The binding of the recombinant proteins produced according to the present invention to cell surface receptors such as the mammalian plasminogen receptor can be determined, for example as described in Example 4 of WO 2021/007612 and Example 4 herein

Finally, the therapeutic efficacy of the recombinant proteins produced according to the present invention can be used according to standard techniques, including those techniques utilised for assessment of the quality of plasminogen and plasmin isolated from human and non-human plasma.

### Compositions

The recombinant plasminogen (or plasmin derived therefrom) can be provided in a pharmaceutically acceptable composition for administration to an individual in need thereof. For example the recombinant proteins made in accordance with the present invention find utility in the treatment of wounds (such as dermal wounds, including abrasions and burns, bone wounds, including bone fractures muscle injury), in providing replacement plasminogen in the context of traumatic injury, in plasminogen replacement therapy where there is a congenital deficiency, treatment of heterotopic ossification and dystrophic calcification.

In some examples, the recombinant plasminogen (or plasmin derived therefrom) as described herein can be administered parenterally, topically, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing a recombinant plasminogen into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

Pharmaceutical compositions of this disclosure are particularly useful for parenteral administration, such as intravenous administration or administration into a body cavity or lumen of an organ or joint. The compositions for administration will commonly comprise a solution of plasminogen dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of plasminogen of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, a recombinant plasminogen made in accordance with the present disclosure will be administered in a manner compatible with the dosage formulation and in such amount as is therapeutically/prophylactically effective.

Sequence information

| SEQ ID | | |
|---|---|---|
| NO: | Description | Sequence |
| 1 | Exemplary | ATGGAACACAAAGAAGTGGTGTTGCTCCTGCTGCTGTTCCTGA |
| | nucleic acid | AGTCCGGCCAGGGCGAGCCCCTGGACGATTACGTGAACACCCA |
| | sequence of | GGGCGCCAGCCTGTTCAGCGTGACCAAGAAACAGCTGGGAGCC |
| | human | GGCAGCATCGAGGAATGCGCCGCCAAGTGCGAAGAGGACGAGG |
| | plasminogen | AATTCACCTGTCGGGCCTTCCAGTACCACAGCAAAGAACAGCA |
| | | GTGCGTGATCATGGCCGAGAACAGAAAGAGCAGCATCATCATC |
| | | AGAATGCGGGACGTGGTGCTGTTCGAGAAGAAGGTGTACCTGA |
| | | GCGAGTGCAAGACCGGCAACGGCAAGAACTACCGGGGCACCAT |
| | | GAGCAAGACCAAGAACGGCATCACCTGTCAGAAGTGGTCCAGC |
| | | ACCAGCCCCCACCGGCCTAGATTTTCTCCAGCCACCCACCCTA |
| | | GCGAGGGCCTGGAAGAGAACTACTGCCGGAACCCCGACAACGA |
| | | CCCTCAGGGCCCTTGGTGCTACACCACCGACCCCGAGAAGAGA |
| | | TACGACTACTGCGACATCCTGGAATGTGAAGAGGAATGCATGC |
| | | ACTGCAGCGGCGAGAACTACGACGGCAAGATCTCCAAGACCAT |
| | | GAGCGGCCTGGAATGCCAGGCTTGGGACAGCCAGTCTCCTCAC |
| | | GCCCACGGCTACATCCCCAGCAAGTTCCCCAACAAGAACCTGA |
| | | AGAAGAATTACTGCAGAAACCCTGACCGCGAGCTGCGGCCCTG |
| | | GTGTTTTACCACCGATCCTAACAAGAGATGGGAGCTGTGCGAT |
| | | ATCCCCCGGTGCACCACACCTCCACCTAGCAGCGGCCCTACCT |
| | | ACCAGTGTCTGAAGGGCACCGGCGAGAATTACAGGGGCAACGT |
| | | GGCCGTGACCGTGTCCGGCCATACCTGCCAGCATTGGAGCGCC |
| | | CAGACCCCCCACACCCACAACAGAACCCCCGAGAACTTCCCCT |
| | | GCAAGAATCTGGACGAGAATTATTGTCGCAACCCCGATGGCAA |
| | | GAGGGCCCCCTGGTGTCACACCACCAACAGCCAGGTGCGCTGG |
| | | GAGTACTGCAAGATCCCCAGCTGCGATAGCAGCCCCGTGTCCA |
| | | CAGAACAGCTGGCCCCTACAGCCCCTCCTGAGCTGACACCTGT |
| | | GGTGCAGGATTGCTACCACGGCGACGGCCAGAGCTACAGAGGC |
| | | ACCAGCAGCACCACCACAACCGGCAAGAAGTGCCAGAGCTGGT |
| | | CCTCCATGACCCCTCACCGGCACCAGAAAACCCCTGAGAATTA |
| | | CCCCAACGCCGGCCTGACCATGAACTACTGTAGAAATCCCGAC |
| | | GCCGACAAGGGACCCTGGTGCTTCACAACAGACCCTTCCGTCA |
| | | GATGGGAATACTGTAATCTGAAGAAGTGCAGCGGCACCGAGGC |
| | | CAGCGTGGTGGCTCCTCCACCAGTGGTGCTGCTGCCCGATGTG |
| | | GAAACCCCCTCCGAAGAGGACTGTATGTTCGGCAATGGCAAGG |
| | | GCTATAGAGGCAAGCGGGCCACCACCGTGACCGGCACACCTTG |
| | | TCAGGATTGGGCCGCTCAGGAACCCCACAGACACAGCATCTTC |
| | | ACCCCAGAGACAAACCCTCGGGCCGGACTGGAAAAAAACTATT |
| | | GTCGGAATCCTGACGGCGACGTGGGAGGACCTTGGTGTTATAC |
| | | AACAAACCCACGGAAGCTGTACGATTACTGTGACGTGCCCCAG |
| | | TGTGCCGCCCCTAGCTTCGATTGTGGCAAGCCCCAGGTGGAAC |
| | | CCAAGAAATGCCCCGGCAGAGTCGTGGGCGGATGTGTGGCCCA |
| | | TCCTCACTCTTGGCCTTGGCAGGTGTCCCTGCGGACCAGATTC |
| | | GGCATGCACTTTTGCGGCGGCACCCTGATCAGCCCCGAGTGGG |
| | | TGCTGACAGCCGCCCACTGTCTGGAAAAGTCCCCCAGACCCAG |
| | | CAGCTACAAAGTGATCCTGGGAGCCCACCAGGAAGTGAACCTG |
| | | GAACCTCACGTGCAGGAAATCGAGGTGTCCAGACTGTTCCTGG |
| | | AACCCACCCGGAAGGATATCGCCCTGCTGAAGCTGAGCAGCCC |
| | | TGCCGTGATCACCGACAAAGTGATTCCCGCCTGCCTGCCCAGC |
| | | CCCAACTATGTGGTGGCCGACAGAACCGAGTGCTTCATCACCG |
| | | GCTGGGGCGAGACACAGGGCACATTTGGAGCCGGCCTGCTGAA |
| | | AGAGGCCCAGCTGCCTGTGATCGAGAACAAAGTGTGCAACCGC |
| | | TACGAGTTCCTGAACGGCAGAGTGCAGAGCACCGAGCTGTGTG |
| | | CCGGACATCTGGCTGGCGGCACAGATAGCTGTCAGGGCGATTC |
| | | TGGCGGCCCTCTCGTGTGCTTCGAGAAGGACAAGTACATCCTG |
| | | CAGGGCGTGACCAGCTGGGGCCTGGGATGTGCCAGACCTAACA |
| | | AGCCCGGCGTGTACGTGCGCGTGTCCAGATTTGTGACCTGGAT |
| | | CGAGGGCGTGATGCGGAACAACTGA |
| 2 | Exemplary | MEHKEVVLLLLLFLKSGQGEPLDDYVNTQGASLFSVTKKQLGA |
| | amino acid | GSIEECAAKCEEDEEFTCRAFQYHSKEQQCVIMAENRKSSIII |
| | sequence of | RMRDVVLFEKKVYLSECKTGNGKNYRGTMSKTKNGITCQKWSS |
| | human | TSPHRPRFSPATHPSEGLEENYCRNPDNDPQGPWCYTTDPEKR |
| | plasminogen | YDYCDILECEEECMHCSGENYDGKISKTMSGLECQAWDSQSPH |
| | Signal | AHGYIPSKFPNKNLKKNYCRNPDRELRPWCFTTDPNKRWELCD |
| | peptide | IPRCTTPPPSSGPTYQCLKGTGENYRGNVAVTVSGHTCQHWSA |
| | underlined. | QTPHTHNRTPENFPCKNLDENYCRNPDGKRAPWCHTTNSQVRW |
| | | EYCKIPSCDSSPVSTEQLAPTAPPELTPVVQDCYHGDGQSYRG |
| | | TSSTTTTGKKCQSWSSMTPHRHQKTPENYPNAGLTMNYCRNPD |
| | | ADKGPWCFTTDPSVRWEYCNLKKCSGTEASVVAPPPVVLLPDV |
| | | ETPSEEDCMFGNGKGYRGKRATTVTGTPCQDWAAQEPHRHSIF |
| | | TPETNPRAGLEKNYCRNPDGDVGGPWCYTTNPRKLYDYCDVPQ |
| | | CAAPSFDCGKPQVEPKKCPGRVVGGCVAHPHSWPWQVSLRTRF |
| | | GMHFCGGTLISPEWVLTAAHCLEKSPRPSSYKVILGAHQEVNL |
| | | EPHVQEIEVSRLFLEPTRKDIALLKLSSPAVITDKVIPACLPS |
| | | PNYVVADRTECFITGWGETQGTFGAGLLKEAQLPVIENKVCNR |
| | | YEFLNGRVQSTELCAGHLAGGTDSCQGDSGGPLVCFEKDKYIL |
| | | QGVTSWGLGCARPNKPGVYVRVSRFVTWIEGVMRNN |
| 3 | Exemplary | ATGCAGATGTCTCCCGCCCTGACCTGCCTGGTGCTGGGCCTGG |
| | nucleic acid | CCCTGGTGTTCGGAGAGGGCTCTGCCGTGCACCACCCACCTAG |
| | sequence of | CTACGTGGCACACCTGGCCTCCGACTTCGGCGTGAGGGTGTTT |
| | recombinant | CAGCAGGTGGCCCAGGCCAGCAAGGATCGCAACGTGGTGTTCA |
| | PAI-1 | GCCCTTATGGCGTGGCCTCCGTGCTGGCCATGCTCCAGCTGAC |
| | Includes | CACAGGAGGAGAGACCCAGCAGCAGATCCAGGCAGCTATGGGC |
| | mutation: | TTCAAGATCGACGATAAGGGAATGGCACCCGCCCTGAGGCACC |
| | Q197C and | TGTACAAGGAGCTGATGGGCCCTTGGAATAAGGACGAGATCAG |
| | G355C | CACCACAGATGCCATCTTTGTGCAGCGCGACCTGAAGCTGGTG |
| | (underlined in | CAGGGCTTCATGCCACACTTCTTTCGGCTGTTCCGGAGCACCG |
| | the sequence | TGAAGCAGGTGGACTTCAGCGAGGTGGAGAGGGCCCGCTTTAT |
| | below) to | CATCAACGATTGGGTGAAGACCCACACAAAGGGCATGATCAGC |
| | improve serpin | AATCTGCTGGGCAAGGGAGCAGTGGATCAGCTGACCAGGCTGG |
| | stability (per | TGCTGGTGAACGCCCTGTACTTCAATGGCTGCTGGAAGACCCC |
| | Chorostowska- | ATTTCCCGACAGCTCCACACACCGGAGACTGTTCCACAAGTCC |
| | Wynimko J | GATGGCTCTACAGTGAGCGTGCCTATGATGGCCCAGACCAACA |
| | et al. (2003) | AGTTCAATTATACAGAGTTTACCACACCTGACGGCCACTACTA |
| | Molecular | TGACATCCTGGAGCTGCCATACCACGGCGACACCCTGAGCATG |
| | Cancer | TTTATCGCCGCCCCTTATGAGAAGGAGGTGCCACTGTCCGCCC |
| | Therapeutics. | TGACAAACATCCTGTCCGCCCAGCTGATCTCTCACTGGAAGGG |
| | 2003;2(1): | CAATATGACCAGGCTGCCAAGGCTGCTGGTGCTGCCTAAGTTC |
| | 19-28. doi: | TCCCTGGAGACAGAGGTGGACCTGCGGAAGCCTCTGGAGAACC |
| | 10.1186/ | TGGGCATGACCGATATGTTCAGACAGTTTCAGGCCGACTTTAC |
| | 1476-4598-2-19) | ATCTCTGAGCGATCAGGAGCCACTGCACGTGGCACAGGCCCTC |
| | | CAGAAGGTGAAGATCGAGGTGAACGAGTCCTGTACCGTGGCCT |
| | | CTAGCTCCACAGCCGTGATCGTGTCTGCCAGGATGGCCCCAGA |
| | | GGAGATCATCATGGATCGGCCCTTCCTGTTTGTGGTGAGACAC |
| | | AATCCAACCGGCACAGTGCTGTTCATGGGCCAGGTCATGGAGC |
| | | CCTGA |
| 4 | Amino acid | MQMSPALTCLVLGLALVFGEGSAVHHPPSYVAHLASDFGVRVF |
| | sequence of | QQVAQASKDRNVVFSPYGVASVLAMLQLTTGGETQQQIQAAMG |
| | recombinant | FKIDDKGMAPALRHLYKELMGPWNKDEISTTDAIFVQRDLKLV |
| | PAI-1, Q197C, | QGFMPHFFRLFRSTVKQVDFSEVERARFIINDWVKTHTKGMIS |
| | G355C. | NLLGKGAVDQLTRLVLVNALYFNGCWKTPFPDSSTHRRLFHKS |
| | Signal peptide | DGSTVSVPMMAQTNKFNYTEFTTPDGHYYDILELPYHGDTLSM |
| | underlined | FIAAPYEKEVPLSALTNILSAQLISHWKGNMTRLPRLLVLPKF |
| | | SLETEVDLRKPLENLGMTDMFRQFQADFTSLSDQEPLHVAQAL |
| | | QKVKIEVNESCTVASSSTAVIVSARMAPEEIIMDRPFLFVVRH |
| | | NPTGTVLFMGQVMEP |
| 5 | Nucleic acid | ATGGAACACAAAGAAGTGGTGTTGCTCCTGCTGCTGTTCCTGA |
| | sequence for | AGTCCGGCCAGGGCGAGCCCCTGGACGATTACGTGAACACCCA |
| | hPIg-IRES2- | GGGCGCCAGCCTGTTCAGCGTGACCAAGAAACAGCTGGGAGCC |
| | PAI-1 | GGCAGCATCGAGGAATGCGCCGCCAAGTGCGAAGAGGACGAGG |
| | expression | AATTCACCTGTCGGGCCTTCCAGTACCACAGCAAAGAACAGCA |
| | cassette | GTGCGTGATCATGGCCGAGAACAGAAAGAGCAGCATCATCATC |
| | (construct for | AGAATGCGGGACGTGGTGCTGTTCGAGAAGAAGGTGTACCTGA |
| | stable | GCGAGTGCAAGACCGGCAACGGCAAGAACTACCGGGGCACCAT |
| | expression of | GAGCAAGACCAAGAACGGCATCACCTGTCAGAAGTGGTCCAGC |
| | plasminogen | ACCAGCCCCCACCGGCCTAGATTTTCTCCAGCCACCCACCCTA |
| | and PAI-1) | GCGAGGGCCTGGAAGAGAACTACTGCCGGAACCCCGACAACGA |
| | | CCCTCAGGGCCCTTGGTGCTACACCACCGACCCCGAGAAGAGA |
| | | TACGACTACTGCGACATCCTGGAATGTGAAGAGGAATGCATGC |
| | | ACTGCAGCGGCGAGAACTACGACGGCAAGATCTCCAAGACCAT |
| | | GAGCGGCCTGGAATGCCAGGCTTGGGACAGCCAGTCTCCTCAC |
| | | GCCCACGGCTACATCCCCAGCAAGTTCCCCAACAAGAACCTGA |
| | | AGAAGAATTACTGCAGAAACCCTGACCGCGAGCTGCGGCCCTG |
| | | GTGTTTTACCACCGATCCTAACAAGAGATGGGAGCTGTGCGAT |
| | | ATCCCCCGGTGCACCACACCTCCACCTAGCAGCGGCCCTACCT |
| | | ACCAGTGTCTGAAGGGCACCGGCGAGAATTACAGGGGCAACGT |
| | | GGCCGTGACCGTGTCCGGCCATACCTGCCAGCATTGGAGCGCC |
| | | CAGACCCCCCACACCCACAACAGAACCCCCGAGAACTTCCCCT |
| | | GCAAGAATCTGGACGAGAATTATTGTCGCAACCCCGATGGCAA |
| | | GAGGGCCCCCTGGTGTCACACCACCAACAGCCAGGTGCGCTGG |
| | | GAGTACTGCAAGATCCCCAGCTGCGATAGCAGCCCCGTGTCCA |
| | | CAGAACAGCTGGCCCCTACAGCCCCTCCTGAGCTGACACCTGT |
| | | GGTGCAGGATTGCTACCACGGCGACGGCCAGAGCTACAGAGGC |
| | | ACCAGCAGCACCACCACAACCGGCAAGAAGTGCCAGAGCTGGT |
| | | CCTCCATGACCCCTCACCGGCACCAGAAAACCCCTGAGAATTA |
| | | CCCCAACGCCGGCCTGACCATGAACTACTGTAGAAATCCCGAC |
| | | GCCGACAAGGGACCCTGGTGCTTCACAACAGACCCTTCCGTCA |
| | | GATGGGAATACTGTAATCTGAAGAAGTGCAGCGGCACCGAGGC |
| | | CAGCGTGGTGGCTCCTCCACCAGTGGTGCTGCTGCCCGATGTG |
| | | GAAACCCCCTCCGAAGAGGACTGTATGTTCGGCAATGGCAAGG |
| | | GCTATAGAGGCAAGCGGGCCACCACCGTGACCGGCACACCTTG |
| | | TCAGGATTGGGCCGCTCAGGAACCCCACAGACACAGCATCTTC |
| | | ACCCCAGAGACAAACCCTCGGGCCGGACTGGAAAAAAACTATT |
| | | GTCGGAATCCTGACGGCGACGTGGGAGGACCTTGGTGTTATAC |
| | | AACAAACCCACGGAAGCTGTACGATTACTGTGACGTGCCCCAG |
| | | TGTGCCGCCCCTAGCTTCGATTGTGGCAAGCCCCAGGTGGAAC |
| | | CCAAGAAATGCCCCGGCAGAGTCGTGGGCGGATGTGTGGCCCA |
| | | TCCTCACTCTTGGCCTTGGCAGGTGTCCCTGCGGACCAGATTC |
| | | GGCATGCACTTTTGCGGCGGCACCCTGATCAGCCCCGAGTGGG |
| | | TGCTGACAGCCGCCCACTGTCTGGAAAAGTCCCCCAGACCCAG |
| | | CAGCTACAAAGTGATCCTGGGAGCCCACCAGGAAGTGAACCTG |
| | | GAACCTCACGTGCAGGAAATCGAGGTGTCCAGACTGTTCCTGG |
| | | AACCCACCCGGAAGGATATCGCCCTGCTGAAGCTGAGCAGCCC |
| | | TGCCGTGATCACCGACAAAGTGATTCCCGCCTGCCTGCCCAGC |
| | | CCCAACTATGTGGTGGCCGACAGAACCGAGTGCTTCATCACCG |
| | | GCTGGGGCGAGACACAGGGCACATTTGGAGCCGGCCTGCTGAA |
| | | AGAGGCCCAGCTGCCTGTGATCGAGAACAAAGTGTGCAACCGC |
| | | TACGAGTTCCTGAACGGCAGAGTGCAGAGCACCGAGCTGTGTG |
| | | CCGGACATCTGGCTGGCGGCACAGATAGCTGTCAGGGCGATTC |
| | | TGGCGGCCCTCTCGTGTGCTTCGAGAAGGACAAGTACATCCTG |
| | | CAGGGCGTGACCAGCTGGGGCCTGGGATGTGCCAGACCTAACA |
| | | AGCCCGGCGTGTACGTGCGCGTGTCCAGATTTGTGACCTGGAT |
| | | CGAGGGCGTGATGCGGAACAACTGAAAGCTTGGTACCGAGCTC |
| | | GGATCCCCCCTCTCCCTCCCCCCCCCCTAACGTTACTGGCCGA |
| | | AGCCGCTTGGAATAAGGCCGGTGTGCGTTTGTCTATATGTTAT |
| | | TTTCCACCATATTGCCGTCTTTTGGCAATGTGAGGGCCCGGAA |
| | | ACCTGGCCCTGTCTTCTTGACGAGCATTCCTAGGGGTCTTTCC |
| | | CCTCTCGCCAAAGGAATGCAAGGTCTGTTGAATGTCGTGAAGG |
| | | AAGCAGTTCCTCTGGAAGCTTCTTGAAGACAAACAACGTCTGT |
| | | AGCGACCCTTTGCAGGCAGCGGAACCCCCCACCTGGCGACAGG |
| | | TGCCTCTGCGGCCAAAAGCCACGTGTATAAGATACACCTGCAA |
| | | AGGCGGCACAACCCCAGTGCCACGTTGTGAGTTGGATAGTTGT |
| | | GGAAAGAGTCAAATGGCTCTCCTCAAGCGTATTCAACAAGGGG |
| | | CTGAAGGATGCCCAGAAGGTACCCCATTGTATGGGATCTGATC |
| | | TGGGGCCTCGGTACACATGCTTTACATGTGTTTAGTCGAGGTT |
| | | AAAAAAACGTCTAGGCCCCCCGAACCACGGGGACGTGGTTTTC |
| | | CTTTGAAAAACACGATGATAATATGCAGATGTCTCCCGCCCTG |
| | | ACCTGCCTGGTGCTGGGCCTGGCCCTGGTGTTCGGAGAGGGCT |
| | | CTGCCGTGCACCACCCACCTAGCTACGTGGCACACCTGGCCTC |
| | | CGACTTCGGCGTGAGGGTGTTTCAGCAGGTGGCCCAGGCCAGC |
| | | AAGGATCGCAACGTGGTGTTCAGCCCTTATGGCGTGGCCTCCG |
| | | TGCTGGCCATGCTCCAGCTGACCACAGGAGGAGAGACCCAGCA |
| | | GCAGATCCAGGCAGCTATGGGCTTCAAGATCGACGATAAGGGA |
| | | ATGGCACCCGCCCTGAGGCACCTGTACAAGGAGCTGATGGGCC |
| | | CTTGGAATAAGGACGAGATCAGCACCACAGATGCCATCTTTGT |
| | | GCAGCGCGACCTGAAGCTGGTGCAGGGCTTCATGCCACACTTC |
| | | TTTCGGCTGTTCCGGAGCACCGTGAAGCAGGTGGACTTCAGCG |
| | | AGGTGGAGAGGGCCCGCTTTATCATCAACGATTGGGTGAAGAC |
| | | CCACACAAAGGGCATGATCAGCAATCTGCTGGGCAAGGGAGCA |
| | | GTGGATCAGCTGACCAGGCTGGTGCTGGTGAACGCCCTGTACT |
| | | TCAATGGCTGCTGGAAGACCCCATTTCCCGACAGCTCCACACA |
| | | CCGGAGACTGTTCCACAAGTCCGATGGCTCTACAGTGAGCGTG |
| | | CCTATGATGGCCCAGACCAACAAGTTCAATTATACAGAGTTTA |
| | | CCACACCTGACGGCCACTACTATGACATCCTGGAGCTGCCATA |
| | | CCACGGCGACACCCTGAGCATGTTTATCGCCGCCCCTTATGAG |
| | | AAGGAGGTGCCACTGTCCGCCCTGACAAACATCCTGTCCGCCC |
| | | AGCTGATCTCTCACTGGAAGGGCAATATGACCAGGCTGCCAAG |
| | | GCTGCTGGTGCTGCCTAAGTTCTCCCTGGAGACAGAGGTGGAC |
| | | CTGCGGAAGCCTCTGGAGAACCTGGGCATGACCGATATGTTCA |
| | | GACAGTTTCAGGCCGACTTTACATCTCTGAGCGATCAGGAGCC |
| | | ACTGCACGTGGCACAGGCCCTCCAGAAGGTGAAGATCGAGGTG |
| | | AACGAGTCCTGTACCGTGGCCTCTAGCTCCACAGCCGTGATCG |
| | | TGTCTGCCAGGATGGCCCCAGAGGAGATCATCATGGATCGGCC |
| | | CTTCCTGTTTGTGGTGAGACACAATCCAACCGGCACAGTGCTG |
| | | TTCATGGGCCAGGTCATGGAGCCCTGA |
| 6 | Exemplary | ATGGAACACAAAGAAGTGGTGTTGCTCCTGCTGCTGTTCCTGA |
| | nucleic acid | AGTCCGGCCAGGGCGAGCCCCTGGACGATTACGTGAACACCCA |
| | sequence of | GGGCGCCAGCCTGTTCAGCGTGACCAAGAAACAGCTGGGAGCC |
| | human glu-Plg | GGCAGCATCGAGGAATGCGCCGCCAAGTGCGAAGAGGACGAGG |
| | | AATTCACCTGTCGGGCCTTCCAGTACCACAGCAAAGAACAGCA |
| | | GTGCGTGATCATGGCCGAGAACAGAAAGAGCAGCATCATCATC |
| | | AGAATGCGGGACGTGGTGCTGTTCGAGAAGAAGGTGTACCTGA |
| | | GCGAGTGCAAGACCGGCAACGGCAAGAACTACCGGGGCACCAT |
| | | GAGCAAGACCAAGAACGGCATCACCTGTCAGAAGTGGTCCAGC |
| | | ACCAGCCCCCACCGGCCTAGATTTTCTCCAGCCACCCACCCTA |
| | | GCGAGGGCCTGGAAGAGAACTACTGCCGGAACCCCGACAACGA |
| | | CCCTCAGGGCCCTTGGTGCTACACCACCGACCCCGAGAAGAGA |
| | | TACGACTACTGCGACATCCTGGAATGTGAAGAGGAATGCATGC |
| | | ACTGCAGCGGCGAGAACTACGACGGCAAGATCTCCAAGACCAT |
| | | GAGCGGCCTGGAATGCCAGGCTTGGGACAGCCAGTCTCCTCAC |
| | | GCCCACGGCTACATCCCCAGCAAGTTCCCCAACAAGAACCTGA |
| | | AGAAGAATTACTGCAGAAACCCTGACCGCGAGCTGCGGCCCTG |
| | | GTGTTTTACCACCGATCCTAACAAGAGATGGGAGCTGTGCGAT |
| | | ATCCCCCGGTGCACCACACCTCCACCTAGCAGCGGCCCTACCT |
| | | ACCAGTGTCTGAAGGGCACCGGCGAGAATTACAGGGGCAACGT |
| | | GGCCGTGACCGTGTCCGGCCATACCTGCCAGCATTGGAGCGCC |
| | | CAGACCCCCCACACCCACAACAGAACCCCCGAGAACTTCCCCT |
| | | GCAAGAATCTGGACGAGAATTATTGTCGCAACCCCGATGGCAA |
| | | GAGGGCCCCCTGGTGTCACACCACCAACAGCCAGGTGCGCTGG |
| | | GAGTACTGCAAGATCCCCAGCTGCGATAGCAGCCCCGTGTCCA |
| | | CAGAACAGCTGGCCCCTACAGCCCCTCCTGAGCTGACACCTGT |
| | | GGTGCAGGATTGCTACCACGGCGACGGCCAGAGCTACAGAGGC |
| | | ACCAGCAGCACCACCACAACCGGCAAGAAGTGCCAGAGCTGGT |
| | | CCTCCATGACCCCTCACCGGCACCAGAAAACCCCTGAGAATTA |
| | | CCCCAACGCCGGCCTGACCATGAACTACTGTAGAAATCCCGAC |
| | | GCCGACAAGGGACCCTGGTGCTTCACAACAGACCCTTCCGTCA |
| | | GATGGGAATACTGTAATCTGAAGAAGTGCAGCGGCACCGAGGC |
| | | CAGCGTGGTGGCTCCTCCACCAGTGGTGCTGCTGCCCGATGTG |
| | | GAAACCCCCTCCGAAGAGGACTGTATGTTCGGCAATGGCAAGG |
| | | GCTATAGAGGCAAGCGGGCCACCACCGTGACCGGCACACCTTG |
| | | TCAGGATTGGGCCGCTCAGGAACCCCACAGACACAGCATCTTC |
| | | ACCCCAGAGACAAACCCTCGGGCCGGACTGGAAAAAAACTATT |
| | | GTCGGAATCCTGACGGCGACGTGGGAGGACCTTGGTGTTATAC |
| | | AACAAACCCACGGAAGCTGTACGATTACTGTGACGTGCCCCAG |
| | | TGTGCCGCCCCTAGCTTCGATTGTGGCAAGCCCCAGGTGGAAC |
| | | CCAAGAAATGCCCCGGCAGAGTCGTGGGGGGATGTGTGGCCCA |
| | | TCCTCACTCTTGGCCTTGGCAGGTGTCCCTGCGGACCAGATTC |
| | | GGCATGCACTTTTGCGGCGGCACCCTGATCAGCCCCGAGTGGG |
| | | TGCTGACAGCCGCCCACTGTCTGGAAAAGTCCCCCAGACCCAG |
| | | CAGCTACAAAGTGATCCTGGGAGCCCACCAGGAAGTGAACCTG |
| | | GAACCTCACGTGCAGGAAATCGAGGTGTCCAGACTGTTCCTGG |
| | | AACCCACCCGGAAGGATATCGCCCTGCTGAAGCTGAGCAGCCC |
| | | TGCCGTGATCACCGACAAAGTGATTCCCGCCTGCCTGCCCAGC |
| | | CCCAACTATGTGGTGGCCGACAGAACCGAGTGCTTCATCACCG |
| | | GCTGGGGCGAGACACAGGGCACATTTGGAGCCGGCCTGCTGAA |
| | | AGAGGCCCAGCTGCCTGTGATCGAGAACAAAGTGTGCAACCGC |
| | | TACGAGTTCCTGAACGGCAGAGTGCAGAGCACCGAGCTGTGTG |
| | | CCGGACATCTGGCTGGCGGCACAGATAGCTGTCAGGGCGATTC |
| | | TGGCGGCCCTCTCGTGTGCTTCGAGAAGGACAAGTACATCCTG |
| | | CAGGGCGTGACCAGCTGGGGCCTGGGATGTGCCAGACCTAACA |
| | | AGCCCGGCGTGTACGTGCGCGTGTCCAGATTTGTGACCTGGAT |
| | | CGAGGGCGTGATGCGGAACAACTGA |
| 7 | Exemplary | MEHKEVVLLLLLFLKSGQGEPLDDYVNTQGASLFSVTKKQLGA |
| | amino acid | GSIEECAAKCEEDEEFTCRAFQYHSKEQQCVIMAENRKSSIII |
| | sequence of | RMRDVVLFEKKVYLSECKTGNGKNYRGTMSKTKNGITCQKWSS |
| | human glu- | TSPHRPRFSPATHPSEGLEENYCRNPDNDPQGPWCYTTDPEKR |
| | Plg Signal | YDYCDILECEEECMHCSGENYDGKISKTMSGLECQAWDSQSPH |
| | peptide | AHGYIPSKFPNKNLKKNYCRNPDRELRPWCFTTDPNKRWELCD |
| | underlined | IPRCTTPPPSSGPTYQCLKGTGENYRGNVAVTVSGHTCQHWSA |
| | | QTPHTHNRTPENFPCKNLDENYCRNPDGKRAPWCHTTNSQVRW |
| | | EYCKIPSCDSSPVSTEQLAPTAPPELTPVVQDCYHGDGQSYRG |
| | | TSSTTTTGKKCQSWSSMTPHRHQKTPENYPNAGLTMNYCRNPD |
| | | ADKGPWCFTTDPSVRWEYCNLKKCSGTEASVVAPPPVVLLPDV |
| | | ETPSEEDCMFGNGKGYRGKRATTVTGTPCQDWAAQEPHRHSIF |
| | | TPETNPRAGLEKNYCRNPDGDVGGPWCYTTNPRKLYDYCDVPQ |
| | | CAAPSFDCGKPQVEPKKCPGRVVGGCVAHPHSWPWQVSLRTRF |
| | | GMHFCGGTLISPEWVLTAAHCLEKSPRPSSYKVILGAHQEVNL |
| | | EPHVQEIEVSRLFLEPTRKDIALLKLSSPAVITDKVIPACLPS |
| | | PNYVVADRTECFITGWGETQGTFGAGLLKEAQLPVIENKVCNR |
| | | YEFLNGRVQSTELCAGHLAGGTDSCQGDSGGPLVCFEKDKYIL |
| | | QGVTSWGLGCARPNKPGVYVRVSRFVTWIEGVMRNN |
| 8 | Exemplary | atggaacacaaagaagtggtgttgctcctgctgctgttcctga |
| | nucleic acid | agtccggccagggcaaggtgtacctgagcgagtgcaagaccgg |
| | sequence of | caacggcaagaactaccggggcaccatgagcaagaccaagaac |
| | human Lys-Plg | ggcatcacctgtcagaagtggtccagcaccagcccccaccggc |
| | | ctagattttctccagccacccaccctagcgagggcctggaaga |
| | | gaactactgccggaaccccgacaacgaccctcagggcccttgg |
| | | tgctacaccaccgaccccgagaagagatacgactactgcgaca |
| | | tcctggaatgtgaagaggaatgcatgcactgcagcggcgagaa |
| | | ctacgacggcaagatctccaagaccatgagcggcctggaatgc |
| | | caggcttgggacagccagtctcctcacgcccacggctacatcc |
| | | ccagcaagttccccaacaagaacctgaagaagaattactgcag |
| | | aaaccctgaccgcgagctgcggccctggtgttttaccaccgat |
| | | cctaacaagagatgggagctgtgcgatatcccccggtgcacca |
| | | cacctccacctagcagcggccctacctaccagtgtctgaaggg |
| | | caccggcgagaattacaggggcaacgtggccgtgaccgtgtcc |
| | | ggccatacctgccagcattggagcgcccagaccccccacaccc |
| | | acaacagaacccccgagaacttcccctgcaagaatctggacga |
| | | gaattattgtcgcaaccccgatggcaagagggccccctggtgt |
| | | cacaccaccaacagccaggtgcgctgggagtactgcaagatcc |
| | | ccagctgcgatagcagccccgtgtccacagaacagctggcccc |
| | | tacagcccctcctgagctgacacctgtggtgcaggattgctac |
| | | cacggcgacggccagagctacagaggcaccagcagcaccacca |
| | | caaccggcaagaagtgccagagctggtcctccatgacccctca |
| | | ccggcaccagaaaacccctgagaattaccccaacgccggcctg |
| | | accatgaactactgtagaaatcccgacgccgacaagggaccct |
| | | ggtgcttcacaacagacccttccgtcagatgggaatactgtaa |
| | | tctgaagaagtgcagcggcaccgaggccagcgtggtggctcct |
| | | ccaccagtggtgctgctgcccgatgtggaaaccccctccgaag |
| | | aggactgtatgttcggcaatggcaagggctatagaggcaagcg |
| | | ggccaccaccgtgaccggcacaccttgtcaggattgggccgct |
| | | caggaaccccacagacacagcatcttcaccccagagacaaacc |
| | | ctcgggccggactggaaaaaaactattgtcggaatcctgacgg |
| | | cgacgtgggaggaccttggtgttatacaacaaacccacggaag |
| | | ctgtacgattactgtgacgtgccccagtgtgccgcccctagct |
| | | tcgattgtggcaagccccaggtggaacccaagaaatgccccgg |
| | | cagagtcgtgggcggatgtgtggcccatcctcactcttggcct |
| | | tggcaggtgtccctgcggaccagattcggcatgcacttttgcg |
| | | gcggcaccctgatcagccccgagtgggtgctgacagccgccca |
| | | ctgtctggaaaagtcccccagacccagcagctacaaagtgatc |
| | | ctgggagcccaccaggaagtgaacctggaacctcacgtgcagg |
| | | aaatcgaggtgtccagactgttcctggaacccacccggaagga |
| | | tatcgccctgctgaagctgagcagccctgccgtgatcaccgac |
| | | aaagtgattcccgcctgcctgcccagccccaactatgtggtgg |
| | | ccgacagaaccgagtgcttcatcaccggctggggcgagacaca |
| | | gggcacatttggagccggcctgctgaaagaggcccagctgcct |
| | | gtgatcgagaacaaagtgtgcaaccgctacgagttcctgaacg |
| | | gcagagtgcagagcaccgagctgtgtgccggacatctggctgg |
| | | cggcacagatagctgtcagggcgattctggcggccctctcgtg |
| | | tgcttcgagaaggacaagtacatcctgcagggcgtgaccagct |
| | | ggggcctgggatgtgccagacctaacaagcccggcgtgtacgt |
| | | gcgcgtgtccagatttgtgacctggatcgagggcgtgatgcgg |
| | | aacaactga |
| 9 | Exemplary | MEHKEVVLLLLLFLKSGQGKVYLSECKTGNGKNYRGTMSKTKN |
| | amino acid | GITCQKWSSTSPHRPRFSPATHPSEGLEENYCRNPDNDPQGPW |
| | sequence of | CYTTDPEKRYDYCDILECEEECMHCSGENYDGKISKTMSGLEC |
| | human Lys-Plg | QAWDSQSPHAHGYIPSKFPNKNLKKNYCRNPDRELRPWCFTTD |
| | Signal | PNKRWELCDIPRCTTPPPSSGPTYQCLKGTGENYRGNVAVTVS |
| | peptide | GHTCQHWSAQTPHTHNRTPENFPCKNLDENYCRNPDGKRAPWC |
| | underlined | HTTNSQVRWEYCKIPSCDSSPVSTEQLAPTAPPELTPVVQDCY |
| | | HGDGQSYRGTSSTTTTGKKCQSWSSMTPHRHQKTPENYPNAGL |
| | | TMNYCRNPDADKGPWCFTTDPSVRWEYCNLKKCSGTEASVVAP |
| | | PPVVLLPDVETPSEEDCMFGNGKGYRGKRATTVTGTPCQDWAA |
| | | QEPHRHSIFTPETNPRAGLEKNYCRNPDGDVGGPWCYTTNPRK |
| | | LYDYCDVPQCAAPSFDCGKPQVEPKKCPGRVVGGCVAHPHSWP |
| | | WQVSLRTRFGMHFCGGTLISPEWVLTAAHCLEKSPRPSSYKVI |
| | | LGAHQEVNLEPHVQEIEVSRLFLEPTRKDIALLKLSSPAVITD |
| | | KVIPACLPSPNYVVADRTECFITGWGETQGTFGAGLLKEAQLP |
| | | VIENKVCNRYEFLNGRVQSTELCAGHLAGGTDSCQGDSGGPLV |
| | | CFEKDKYILQGVTSWGLGCARPNKPGVYVRVSRFVTWIEGVMR |
| | | NN |
| 10 | Exemplary | ATGGAACACAAAGAAGTGGTGTTGCTCCTGCTGCTGTTCCTGA |
| | nucleic acid | AGTCCGGCCAGGGCGATTGCTACCACGGCGACGGCCAGAGCTA |
| | sequence of | CAGAGGCACCAGCAGCACCACCACAACCGGCAAGAAGTGCCAG |
| | human Midi-Plg | AGCTGGTCCTCCATGACCCCTCACCGGCACCAGAAAACCCCTG |
| | | AGAATTACCCCAACGCCGGCCTGACCATGAACTACTGTAGAAA |
| | | TCCCGACGCCGACAAGGGACCCTGGTGCTTCACAACAGACCCT |
| | | TCCGTCAGATGGGAATACTGTAATCTGAAGAAGTGCAGCGGCA |
| | | CCGAGGCCAGCGTGGTGGCTCCTCCACCAGTGGTGCTGCTGCC |
| | | CGATGTGGAAACCCCCTCCGAAGAGGACTGTATGTTCGGCAAT |
| | | GGCAAGGGCTATAGAGGCAAGCGGGCCACCACCGTGACCGGCA |
| | | CACCTTGTCAGGATTGGGCCGCTCAGGAACCCCACAGACACAG |
| | | CATCTTCACCCCAGAGACAAACCCTCGGGCCGGACTGGAAAAA |
| | | AACTATTGTCGGAATCCTGACGGCGACGTGGGAGGACCTTGGT |
| | | GTTATACAACAAACCCACGGAAGCTGTACGATTACTGTGACGT |
| | | GCCCCAGTGTGCCGCCCCTAGCTTCGATTGTGGCAAGCCCCAG |
| | | GTGGAACCCAAGAAATGCCCCGGCAGAGTCGTGGGCGGATGTG |
| | | TGGCCCATCCTCACTCTTGGCCTTGGCAGGTGTCCCTGCGGAC |
| | | CAGATTCGGCATGCACTTTTGCGGCGGCACCCTGATCAGCCCC |
| | | GAGTGGGTGCTGACAGCCGCCCACTGTCTGGAAAAGTCCCCCA |
| | | GACCCAGCAGCTACAAAGTGATCCTGGGAGCCCACCAGGAAGT |
| | | GAACCTGGAACCTCACGTGCAGGAAATCGAGGTGTCCAGACTG |
| | | TTCCTGGAACCCACCCGGAAGGATATCGCCCTGCTGAAGCTGA |
| | | GCAGCCCTGCCGTGATCACCGACAAAGTGATTCCCGCCTGCCT |
| | | GCCCAGCCCCAACTATGTGGTGGCCGACAGAACCGAGTGCTTC |
| | | ATCACCGGCTGGGGCGAGACACAGGGCACATTTGGAGCCGGCC |
| | | TGCTGAAAGAGGCCCAGCTGCCTGTGATCGAGAACAAAGTGTG |
| | | CAACCGCTACGAGTTCCTGAACGGCAGAGTGCAGAGCACCGAG |
| | | CTGTGTGCCGGACATCTGGCTGGCGGCACAGATAGCTGTCAGG |
| | | GCGATTCTGGCGGCCCTCTCGTGTGCTTCGAGAAGGACAAGTA |
| | | CATCCTGCAGGGCGTGACCAGCTGGGGCCTGGGATGTGCCAGA |
| | | CCTAACAAGCCCGGCGTGTACGTGCGCGTGTCCAGATTTGTGA |
| | | CCTGGATCGAGGGCGTGATGCGGAACAACTGA |
| 11 | Exemplary | MEHKEVVLLLLLFLKSGQGDCYHGDGQSYRGTSSTTTTGKKCQ |
| | amino acid | SWSSMTPHRHQKTPENYPNAGLTMNYCRNPDADKGPWCFTTDP |
| | sequence of | SVRWEYCNLKKCSGTEASVVAPPPVVLLPDVETPSEEDCMFGN |
| | human Midi- | GKGYRGKRATTVTGTPCQDWAAQEPHRHSIFTPETNPRAGLEK |
| | Plg Signal | NYCRNPDGDVGGPWCYTTNPRKLYDYCDVPQCAAPSFDCGKPQ |
| | peptide | VEPKKCPGRVVGGCVAHPHSWPWQVSLRTRFGMHFCGGTLISP |
| | underlined | EWVLTAAHCLEKSPRPSSYKVILGAHQEVNLEPHVQEIEVSRL |
| | | FLEPTRKDIALLKLSSPAVITDKVIPACLPSPNYVVADRTECF |
| | | ITGWGETQGTFGAGLLKEAQLPVIENKVCNRYEFLNGRVQSTE |
| | | LCAGHLAGGTDSCQGDSGGPLVCFEKDKYILQGVTSWGLGCAR |
| | | PNKPGVYVRVSRFVTWIEGVMRNN |
| 12 | Exemplary | atggaacacaaagaagtggtgttgctcctgctgctgttcctga |
| | nucleic acid | agtccggccagggcgaggactgtatgttcggcaatggcaaggg |
| | sequence of | ctatagaggcaagcgggccaccaccgtgaccggcacaccttgt |
| | human mini-Plg | caggattgggccgctcaggaaccccacagacacagcatcttca |
| | | ccccagagacaaaccctcgggccggactggaaaaaaactattg |
| | | tcggaatcctgacggcgacgtgggaggaccttggtgttataca |
| | | acaaacccacggaagctgtacgattactgtgacgtgccccagt |
| | | gtgccgcccctagcttcgattgtggcaagccccaggtggaacc |
| | | caagaaatgccccggcagagtcgtgggcggatgtgtggcccat |
| | | cctcactcttggccttggcaggtgtccctgcggaccagattcg |
| | | gcatgcacttttgcggcggcaccctgatcagccccgagtgggt |
| | | gctgacagccgcccactgtctggaaaagtcccccagacccagc |
| | | agctacaaagtgatcctgggagcccaccaggaagtgaacctgg |
| | | aacctcacgtgcaggaaatcgaggtgtccagactgttcctgga |
| | | acccacccggaaggatatcgccctgctgaagctgagcagccct |
| | | gccgtgatcaccgacaaagtgattcccgcctgcctgcccagcc |
| | | ccaactatgtggtggccgacagaaccgagtgcttcatcaccgg |
| | | ctggggcgagacacagggcacatttggagccggcctgctgaaa |
| | | gaggcccagctgcctgtgatcgagaacaaagtgtgcaaccgct |
| | | acgagttcctgaacggcagagtgcagagcaccgagctgtgtgc |
| | | cggacatctggctggcggcacagatagctgtcagggcgattct |
| | | ggcggccctctcgtgtgcttcgagaaggacaagtacatcctgc |
| | | agggcgtgaccagctggggcctgggatgtgccagacctaacaa |
| | | gcccggcgtgtacgtgcgcgtgtccagatttgtgacctggatc |
| | | gagggcgtgatgcggaacaactga |
| 13 | Exemplary | MEHKEVVLLLLLFLKSGQGEDCMFGNGKGYRGKRATTVTGTPC |
| | amino acid | QDWAAQEPHRHSIFTPETNPRAGLEKNYCRNPDGDVGGPWCYT |
| | sequence of | TNPRKLYDYCDVPQCAAPSFDCGKPQVEPKKCPGRVVGGCVAH |
| | human mini- | PHSWPWQVSLRTRFGMHFCGGTLISPEWVLTAAHCLEKSPRPS |
| | Plg Signal | SYKVILGAHQEVNLEPHVQEIEVSRLFLEPTRKDIALLKLSSP |
| | peptide | AVITDKVIPACLPSPNYVVADRTECFITGWGETQGTFGAGLLK |
| | underlined | EAQLPVIENKVCNRYEFLNGRVQSTELCAGHLAGGTDSCQGDS |
| | | GGPLVCFEKDKYILQGVTSWGLGCARPNKPGVYVRVSRFVTWI |
| | | EGVMRNN |
| 14 | Exemplary | atggaacacaaagaagtggtgttgctcctgctgctgttcctga |
| | nucleic acid | agtccggccagggcgcccctagcttcgattgtggcaagcccca |
| | sequence of | ggtggaacccaagaaatgccccggcagagtcgtgggcggatgt |
| | human | gtggcccatcctcactcttggccttggcaggtgtccctgcgga |
| | micro-Plg | ccagattcggcatgcacttttgcggcggcaccctgatcagccc |
| | | cgagtgggtgctgacagccgcccactgtctggaaaagtccccc |
| | | agacccagcagctacaaagtgatcctgggagcccaccaggaag |
| | | tgaacctggaacctcacgtgcaggaaatcgaggtgtccagact |
| | | gttcctggaacccacccggaaggatatcgccctgctgaagctg |
| | | agcagccctgccgtgatcaccgacaaagtgattcccgcctgcc |
| | | tgcccagccccaactatgtggtggccgacagaaccgagtgctt |
| | | catcaccggctggggcgagacacagggcacatttggagccggc |
| | | ctgctgaaagaggcccagctgcctgtgatcgagaacaaagtgt |
| | | gcaaccgctacgagttcctgaacggcagagtgcagagcaccga |
| | | gctgtgtgccggacatctggctggcggcacagatagctgtcag |
| | | ggcgattctggcggccctctcgtgtgcttcgagaaggacaagt |
| | | acatcctgcagggcgtgaccagctggggcctgggatgtgccag |
| | | acctaacaagcccggcgtgtacgtgcgcgtgtccagatttgtg |
| | | acctggatcgagggcgtgatgcggaacaactga |
| 15 | Exemplary | MEHKEVVLLLLLFLKSGQGAPSFDCGKPQVEPKKCPGRVVGGC |
| | amino acid | VAHPHSWPWQVSLRTRFGMHFCGGTLISPEWVLTAAHCLEKSP |
| | sequence of | RPSSYKVILGAHQEVNLEPHVQEIEVSRLFLEPTRKDIALLKL |
| | human | SSPAVITDKVIPACLPSPNYVVADRTECFITGWGETQGTFGAG |
| | micro-Plg | LLKEAQLPVIENKVCNRYEFLNGRVQSTELCAGHLAGGTDSCQ |
| | Signal peptide | GDSGGPLVCFEKDKYILQGVTSWGLGCARPNKPGVYVRVSRFV |
| | underlined | TWIEGVMRNN |
| 16 | Exemplary | EPLDDYVNTQGASLFSVTKKQLGAGSIEECAAKCEEDEEFTCR |
| | amino acid | AFQYHSKEQQCVIMAENRKSSIIIRMRDVVLFEKKVYLSECKT |
| | sequence of | GNGKNYRGTMSKTKNGITCQKWSSTSPHRPRFSPATHPSEGLE |
| | human Glu-Plg | ENYCRNPDNDPQGPWCYTTDPEKRYDYCDILECEEECMHCSGE |
| | with signal | NYDGKISKTMSGLECQAWDSQSPHAHGYIPSKFPNKNLKKNYC |
| | peptide | RNPDRELRPWCFTTDPNKRWELCDIPRCTTPPPSSGPTYQCLK |
| | removed | GTGENYRGNVAVTVSGHTCQHWSAQTPHTHNRTPENFPCKNLD |
| | | ENYCRNPDGKRAPWCHTTNSQVRWEYCKIPSCDSSPVSTEQLA |
| | | PTAPPELTPVVQDCYHGDGQSYRGTSSTTTTGKKCQSWSSMTP |
| | | HRHQKTPENYPNAGLTMNYCRNPDADKGPWCFTTDPSVRWEYC |
| | | NLKKCSGTEASVVAPPPVVLLPDVETPSEEDCMFGNGKGYRGK |
| | | RATTVTGTPCQDWAAQEPHRHSIFTPETNPRAGLEKNYCRNPD |
| | | GDVGGPWCYTTNPRKLYDYCDVPQCAAPSFDCGKPQVEPKKCP |
| | | GRVVGGCVAHPHSWPWQVSLRTRFGMHFCGGTLISPEWVLTAA |
| | | HCLEKSPRPSSYKVILGAHQEVNLEPHVQEIEVSRLFLEPTRK |
| | | DIALLKLSSPAVITDKVIPACLPSPNYVVADRTECFITGWGET |
| | | QGTFGAGLLKEAQLPVIENKVCNRYEFLNGRVQSTELCAGHLA |
| | | GGTDSCQGDSGGPLVCFEKDKYILQGVTSWGLGCARPNKPGVY |
| | | VRVSRFVTWIEGVMRNN |
| 17 | Exemplary | KVYLSECKTGNGKNYRGTMSKTKNGITCQKWSSTSPHRPRFSP |
| | amino acid | ATHPSEGLEENYCRNPDNDPQGPWCYTTDPEKRYDYCDILECE |
| | sequence of | EECMHCSGENYDGKISKTMSGLECQAWDSQSPHAHGYIPSKFP |
| | human Lys-Plg | NKNLKKNYCRNPDRELRPWCFTTDPNKRWELCDIPRCTTPPPS |
| | with signal | SGPTYQCLKGTGENYRGNVAVTVSGHTCQHWSAQTPHTHNRTP |
| | peptide | ENFPCKNLDENYCRNPDGKRAPWCHTTNSQVRWEYCKIPSCDS |
| | removed | SPVSTEQLAPTAPPELTPVVQDCYHGDGQSYRGTSSTTTTGKK |
| | | CQSWSSMTPHRHQKTPENYPNAGLTMNYCRNPDADKGPWCFTT |
| | | DPSVRWEYCNLKKCSGTEASVVAPPPVVLLPDVETPSEEDCMF |
| | | GNGKGYRGKRATTVTGTPCQDWAAQEPHRHSIFTPETNPRAGL |
| | | EKNYCRNPDGDVGGPWCYTTNPRKLYDYCDVPQCAAPSFDCGK |
| | | PQVEPKKCPGRVVGGCVAHPHSWPWQVSLRTRFGMHFCGGTLI |
| | | SPEWVLTAAHCLEKSPRPSSYKVILGAHQEVNLEPHVQEIEVS |
| | | RLFLEPTRKDIALLKLSSPAVITDKVIPACLPSPNYVVADRTE |
| | | CFITGWGETQGTFGAGLLKEAQLPVIENKVCNRYEFLNGRVQS |
| | | TELCAGHLAGGTDSCQGDSGGPLVCFEKDKYILQGVTSWGLGC |
| | | ARPNKPGVYVRVSRFVTWIEGVMRNN |
| 18 | Exemplary | DCYHGDGQSYRGTSSTTTTGKKCQSWSSMTPHRHQKTPENYPN |
| | amino acid | AGLTMNYCRNPDADKGPWCFTTDPSVRWEYCNLKKCSGTEASV |
| | sequence of | VAPPPVVLLPDVETPSEEDCMFGNGKGYRGKRATTVTGTPCQD |
| | human midi-Plg | WAAQEPHRHSIFTPETNPRAGLEKNYCRNPDGDVGGPWCYTTN |
| | with signal | PRKLYDYCDVPQCAAPSFDCGKPQVEPKKCPGRVVGGCVAHPH |
| | peptide | SWPWQVSLRTRFGMHFCGGTLISPEWVLTAAHCLEKSPRPSSY |
| | removed | KVILGAHQEVNLEPHVQEIEVSRLFLEPTRKDIALLKLSSPAV |
| | | ITDKVIPACLPSPNYVVADRTECFITGWGETQGTFGAGLLKEA |
| | | QLPVIENKVCNRYEFLNGRVQSTELCAGHLAGGTDSCQGDSGG |
| | | PLVCFEKDKYILQGVTSWGLGCARPNKPGVYVRVSRFVTWIEG |
| | | VMRNN |
| 19 | Exemplary | EDCMFGNGKGYRGKRATTVTGTPCQDWAAQEPHRHSIFTPETN |
| | amino acid | PRAGLEKNYCRNPDGDVGGPWCYTTNPRKLYDYCDVPQCAAPS |
| | sequence of | FDCGKPQVEPKKCPGRVVGGCVAHPHSWPWQVSLRTRFGMHFC |
| | human mini-Plg | GGTLISPEWVLTAAHCLEKSPRPSSYKVILGAHQEVNLEPHVQ |
| | with signal | EIEVSRLFLEPTRKDIALLKLSSPAVITDKVIPACLPSPNYVV |
| | peptide | ADRTECFITGWGETQGTFGAGLLKEAQLPVIENKVCNRYEFLN |
| | removed | GRVQSTELCAGHLAGGTDSCQGDSGGPLVCFEKDKYILQGVTS |
| | | WGLGCARPNKPGVYVRVSRFVTWIEGVMRNN |
| 20 | Exemplary | APSFDCGKPQVEPKKCPGRVVGGCVAHPHSWPWQVSLRTRFGM |
| | amino acid | HFCGGTLISPEWVLTAAHCLEKSPRPSSYKVILGAHQEVNLEP |
| | sequence of | HVQEIEVSRLFLEPTRKDIALLKLSSPAVITDKVIPACLPSPN |
| | human micro- | YVVADRTECFITGWGETQGTFGAGLLKEAQLPVIENKVCNRYE |
| | Plg with | FLNGRVQSTELCAGHLAGGTDSCQGDSGGPLVCFEKDKYILQG |
| | signal peptide | VTSWGLGCARPNKPGVYVRVSRFVTWIEGVMRNN |
| | removed | |
| 21 | Exemplary | ATGGAACACAAAGAAGTGGTGTTGCTCCTGCTGCTGTTCCTGA |
| | nucleotide | AGTCCGGCCAGGGCGAGCCCCTGGACGATTACGTGAACACCCA |
| | sequence of | GGGCGCCAGCCTGTTCAGCGTGACCAAGAAACAGCTGGGAGCC |
| | Plasminogen- | GGCAGCATCGAGGAATGCGCCGCCAAGTGCGAAGAGGACGAGG |
| | linker-Fc | AATTCACCTGTCGGGCCTTCCAGTACCACAGCAAAGAACAGCA |
| | fusion | GTGCGTGATCATGGCCGAGAACAGAAAGAGCAGCATCATCATC |
| | | AGAATGCGGGACGTGGTGCTGTTCGAGAAGAAGGTGTACCTGA |
| | | GCGAGTGCAAGACCGGCAACGGCAAGAACTACCGGGGCACCAT |
| | | GAGCAAGACCAAGAACGGCATCACCTGTCAGAAGTGGTCCAGC |
| | | ACCAGCCCCCACCGGCCTAGATTTTCTCCAGCCACCCACCCTA |
| | | GCGAGGGCCTGGAAGAGAACTACTGCCGGAACCCCGACAACGA |
| | | CCCTCAGGGCCCTTGGTGCTACACCACCGACCCCGAGAAGAGA |
| | | TACGACTACTGCGACATCCTGGAATGTGAAGAGGAATGCATGC |
| | | ACTGCAGCGGCGAGAACTACGACGGCAAGATCTCCAAGACCAT |
| | | GAGCGGCCTGGAATGCCAGGCTTGGGACAGCCAGTCTCCTCAC |
| | | GCCCACGGCTACATCCCCAGCAAGTTCCCCAACAAGAACCTGA |
| | | AGAAGAATTACTGCAGAAACCCTGACCGCGAGCTGCGGCCCTG |
| | | GTGTTTTACCACCGATCCTAACAAGAGATGGGAGCTGTGCGAT |
| | | ATCCCCCGGTGCACCACACCTCCACCTAGCAGCGGCCCTACCT |
| | | ACCAGTGTCTGAAGGGCACCGGCGAGAATTACAGGGGCAACGT |
| | | GGCCGTGACCGTGTCCGGCCATACCTGCCAGCATTGGAGCGCC |
| | | CAGACCCCCCACACCCACAACAGAACCCCCGAGAACTTCCCCT |
| | | GCAAGAATCTGGACGAGAATTATTGTCGCAACCCCGATGGCAA |
| | | GAGGGCCCCCTGGTGTCACACCACCAACAGCCAGGTGCGCTGG |
| | | GAGTACTGCAAGATCCCCAGCTGCGATAGCAGCCCCGTGTCCA |
| | | CAGAACAGCTGGCCCCTACAGCCCCTCCTGAGCTGACACCTGT |
| | | GGTGCAGGATTGCTACCACGGCGACGGCCAGAGCTACAGAGGC |
| | | ACCAGCAGCACCACCACAACCGGCAAGAAGTGCCAGAGCTGGT |
| | | CCTCCATGACCCCTCACCGGCACCAGAAAACCCCTGAGAATTA |
| | | CCCCAACGCCGGCCTGACCATGAACTACTGTAGAAATCCCGAC |
| | | GCCGACAAGGGACCCTGGTGCTTCACAACAGACCCTTCCGTCA |
| | | GATGGGAATACTGTAATCTGAAGAAGTGCAGCGGCACCGAGGC |
| | | CAGCGTGGTGGCTCCTCCACCAGTGGTGCTGCTGCCCGATGTG |
| | | GAAACCCCCTCCGAAGAGGACTGTATGTTCGGCAATGGCAAGG |
| | | GCTATAGAGGCAAGCGGGCCACCACCGTGACCGGCACACCTTG |
| | | TCAGGATTGGGCCGCTCAGGAACCCCACAGACACAGCATCTTC |
| | | ACCCCAGAGACAAACCCTCGGGCCGGACTGGAAAAAAACTATT |
| | | GTCGGAATCCTGACGGCGACGTGGGAGGACCTTGGTGTTATAC |
| | | AACAAACCCACGGAAGCTGTACGATTACTGTGACGTGCCCCAG |
| | | TGTGCCGCCCCTAGCTTCGATTGTGGCAAGCCCCAGGTGGAAC |
| | | CCAAGAAATGCCCCGGCAGAGTCGTGGGCGGATGTGTGGCCCA |
| | | TCCTCACTCTTGGCCTTGGCAGGTGTCCCTGCGGACCAGATTC |
| | | GGCATGCACTTTTGCGGCGGCACCCTGATCAGCCCCGAGTGGG |
| | | TGCTGACAGCCGCCCACTGTCTGGAAAAGTCCCCCAGACCCAG |
| | | CAGCTACAAAGTGATCCTGGGAGCCCACCAGGAAGTGAACCTG |
| | | GAACCTCACGTGCAGGAAATCGAGGTGTCCAGACTGTTCCTGG |
| | | AACCCACCCGGAAGGATATCGCCCTGCTGAAGCTGAGCAGCCC |
| | | TGCCGTGATCACCGACAAAGTGATTCCCGCCTGCCTGCCCAGC |
| | | CCCAACTATGTGGTGGCCGACAGAACCGAGTGCTTCATCACCG |
| | | GCTGGGGCGAGACACAGGGCACATTTGGAGCCGGCCTGCTGAA |
| | | AGAGGCCCAGCTGCCTGTGATCGAGAACAAAGTGTGCAACCGC |
| | | TACGAGTTCCTGAACGGCAGAGTGCAGAGCACCGAGCTGTGTG |
| | | CCGGACATCTGGCTGGCGGCACAGATAGCTGTCAGGGCGATTC |
| | | TGGCGGCCCTCTCGTGTGCTTCGAGAAGGACAAGTACATCCTG |
| | | CAGGGCGTGACCAGCTGGGGCCTGGGATGTGCCAGACCTAACA |
| | | AGCCCGGCGTGTACGTGCGCGTGTCCAGATTTGTGACCTGGAT |
| | | CGAGGGCGTGATGCGGAACAACGGCCAGGCCGGCCAAGCTTCC |
| | | GACAAAACTCACACATGCCCACCGTGCCCAGCACCTGAACTCC |
| | | TGGGGGGACCGTCAGTCTTCCTCTTCCCCCCAAAACCCAAGGA |
| | | CACCCTCATGATCTCCCGGACCCCTGAGGTCACATGCGTGGTG |
| | | GTGGACGTGAGCCACGAAGACCCTGAGGTCAAGTTCAACTGGT |
| | | ACGTGGACGGCGTGGAGGTGCATAATGCCAAGACAAAGCCGCG |
| | | GGAGGAGCAGTACAACAGCACGTACCGTGTGGTCAGCGTCCTC |
| | | ACCGTCCTGCACCAGGACTGGCTGAATGGCAAGGAGTACAAGT |
| | | GCAAGGTCTCCAACAAAGCCCTCCCAGCCCCCATCGAGAAAAC |
| | | CATCTCCAAAGCCAAAGGGCAGCCCCGAGAACCACAGGTGTAC |
| | | ACCCTGCCCCCATCCCGGGAGGAGATGACCAAGAACCAGGTCA |
| | | GCCTGACCTGCCTGGTCAAAGGCTTCTATCCCAGCGACATCGC |
| | | CGTGGAGTGGGAGAGCAATGGGCAGCCGGAGAACAACTACAAG |
| | | ACCACGCCTCCCGTGCTGGACTCCGACGGCTCCTTCTTCCTCT |
| | | ACAGCAAGCTCACCGTGGACAAGAGCAGGTGGCAGCAGGGGAA |
| | | CGTCTTCTCATGCTCCGTGATGCACGAGGCTCTGCACAACCAC |
| | | TACACGCAGAAGAGCCTCTCCCTGTCTCCGGGTAAATGA |
| 22 | Exemplary | MEHKEVVLLLLLFLKSGQGEPLDDYVNTQGASLFSVTKKQLGA |
| | amino acid | GSIEECAAKCEEDEEFTCRAFQYHSKEQQCVIMAENRKSSIII |
| | sequence of | RMRDVVLFEKKVYLSECKTGNGKNYRGTMSKTKNGITCQKWSS |
| | recombinant | TSPHRPRFSPATHPSEGLEENYCRNPDNDPQGPWCYTTDPEKR |
| | Plasminogen- | YDYCDILECEEECMHCSGENYDGKISKTMSGLECQAWDSQSPH |
| | linker-Fc | AHGYIPSKFPNKNLKKNYCRNPDRELRPWCFTTDPNKRWELCD |
| | fusion | IPRCTTPPPSSGPTYQCLKGTGENYRGNVAVTVSGHTCQHWSA |
| | | QTPHTHNRTPENFPCKNLDENYCRNPDGKRAPWCHTTNSQVRW |
| | | EYCKIPSCDSSPVSTEQLAPTAPPELTPVVQDCYHGDGQSYRG |
| | | TSSTTTTGKKCQSWSSMTPHRHQKTPENYPNAGLTMNYCRNPD |
| | | ADKGPWCFTTDPSVRWEYCNLKKCSGTEASVVAPPPVVLLPDV |
| | | ETPSEEDCMFGNGKGYRGKRATTVTGTPCQDWAAQEPHRHSIF |
| | | TPETNPRAGLEKNYCRNPDGDVGGPWCYTTNPRKLYDYCDVPQ |
| | | CAAPSFDCGKPQVEPKKCPGRVVGGCVAHPHSWPWQVSLRTRF |
| | | GMHFCGGTLISPEWVLTAAHCLEKSPRPSSYKVILGAHQEVNL |
| | | EPHVQEIEVSRLFLEPTRKDIALLKLSSPAVITDKVIPACLPS |
| | | PNYVVADRTECFITGWGETQGTFGAGLLKEAQLPVIENKVCNR |
| | | YEFLNGRVQSTELCAGHLAGGTDSCQGDSGGPLVCFEKDKYIL |
| | | QGVTSWGLGCARPNKPGVYVRVSRFVTWIEGVMRNNGQAGQAS |
| | | DKTHTCPPCPAPELLGGPSVFLFPPKPKDTLMISRTPEVTCVV |
| | | VDVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYK |
| | | TTPPVLDSDGSFFLYSKLTVDKSRWQQGNVFSCSVMHEALHNH |
| | | YTQKSLSLSPGK |
| 23 | Exemplary | GQAGQAS |
| | amino acid | |
| | sequence of | |
| | linker (QA | |
| | linker) | |
| 24 | Exemplary | DKTHTCPPCPAPELLGGPSVFLFPPKPKDTLMISRTPEVTCVV |
| | amino acid | VDVSHEDPEVKFNWYVDGVEVHNAKTKPREEQYNSTYRVVSVL |
| | sequence of | TVLHQDWLNGKEYKCKVSNKALPAPIEKTISKAKGQPREPQVY |
| | an Fc region | TLPPSREEMTKNQVSLTCLVKGFYPSDIAVEWESNGQPENNYK |
| | of an | TTPPVLDSDGSFFLYSKLTVDKSRWQQGNVFSCSVMHEALHNH |
| | antibody | YTQKSLSLSPGK |
| 25 | Alternative | PAPELLGGSSVFLFPPKPKDTLMISRTPEVTCVVVDVSHEDPE |
| | amino acid | VKFNWYVDGVEVHNAKTKPREEQYNSTYRVVSVLTVLHQDWLN |
| | sequence of | GKEYKCKVSNKALPAPIEKTISKAKGQPREPQVYTLPPSRDEL |
| | Fc region of | TKNQVNLTCLVKGFYPSDIAVEWESNGQPENNYKTTPPVLDSD |
| | an antibody | GSFFLNSTLTVDKSRWQQGNVFSCSVMHEALHNHYTQKSLSLS |
| | | PGK |
| 26 | Alternative | PAPELLGGSSVFLFPPKPKDTLMISRTPEVTCVVVDVSHEDPE |
| | amino acid | VKFNWYVDGVEVHNAKTKPREEQYNSTYRVVSVLTVLHQDWLN |
| | sequence of | GKEYKCKVSNKALPAPIEKTISKAKGQPREPQVYTLPPSRDEL |
| | Fc region of | TKNQVSLTCLVKGFYPSDIAVEWESNGQPENNYKTTPPVLDSD |
| | an antibody | GSFFLYSKLTVDKSRWQQGNVFSCSVMHEALHNHYTQKSLSLS |
| | | PGK |
| 27 | Fc(IgG1) (EU | PAPELLGGPSVFLFPPKPKDTLMISRTPEVTCWVDVSHEDPEV |
| | numbering | KFNWYVDGVEVHNAKTKPREEQYNSTYRVVSVLTVLHQDWLNG |
| | 230-447) | KEYKCKVSNKALPAPIEKTISKAKGQPREPQVYTLPPSRDELT |
| | | KNQVSLTCLVKGFYPSDIAVEWESNGQPENNYKTTPPVLDSDG |
| | | SFFLYSKLTVDKSRWQQGNVFSCSVMHEALHNHYTQKSLSLSP |
| | | GK |
| 28 | Fc(IgG2) (EU | PAPPVAGPSVFLFPPKPKDTLMISRTPEVTCVVVDVSHEDPEV |
| | numbering | QFNWYVDGVEVHNAKTKPREEQFNSTFRVVSVLTVVHQDWLNG |
| | 230-447) | KEYKCKVSNKGLPAPIEKTISKTKGQPREPQVYTLPPSREEMT |
| | | KNQVSLTCLVKGFYPSDIAVEWESNGQPENNYKTTPPMLDSDG |
| | | SFFLYSKLTVDKSRWQQGNVFSCSVMHEALHNHYTQKSLSLSP |
| | | GK |
| 29 | Fc(IgG3) (EU | PAPELLGGPSVFLFPPKPKDTLMISRTPEVTCVVVDVSHEDPE |
| | numbering | VQFKWYVDGVEVHNAKTKPREEQYNSTFRVVSVLTVLHQDWLN |
| | 230-447) | GKEYKCKVSNKALPAPIEKTISKTKGQPREPQVYTLPPSREEM |
| | | TKNQVSLTCLVKGFYPSDIAVEWESSGQPENNYNTTPPMLDSD |
| | | GSFFLYSKLTVDKSRWQQGNIFSCSVMHEALHNRFTQKSLSLS |
| | | PGK |
| 30 | Fc(IgG4) (EU | PAPEFLGGPSVFLFPPKPKDTLMISRTPEVTCVVVDVSQEDPE |
| | numbering | VQFNWYVDGVEVHNAKTKPREEQFNSTYRVVSVLTVLHQDWLN |
| | 230-447) | GKEYKCKVSNKGLPSSIEKTISKAKGQPREPQVYTLPPSQEEM |
| | | TKNQVSLTCLVKGFYPSDIAVEWESNGQPENNYKTTPPVLDSD |
| | | GSFFLYSRLTVDKSRWQEGNVFSCSVMHEALHNHYTQKSLSLS |
| | | LGK |
| 31 | Fc(IgG1-238S) | PAPELLGGSSVFLFPPKPKDTLMISRTPEVTCVVVDVSHEDPE |
| | (EU numbering | VKFNWYVDGVEVHNAKTKPREEQYNSTYRVVSVLTVLHQDWLN |
| | 230-447) | GKEYKCKVSNKALPAPIEKTISKAKGQPREPQVYTLPPSRDEL |
| | | TKNQVSLTCLVKGFYPSDIAVEWESNGQPENNYKTTPPVLDSD |
| | | GSFFLYSKLTVDKSRWQQGNVFSCSVMHEALHNHYTQKSLSLS |
| | | PGK |
| 32 | Fc(IgG2- | PAPELLGGPSVFLFPPKPKDTLMISRTPEVTCWVDVSHEDPEV |
| | 233E/234L/ | QFNWYVDGVEVHNAKTKPREEQFNSTFRVVSVLTVVHQDWLNG |
| | 235L/236G) | KEYKCKVSNKGLPAPIEKTISKTKGQPREPQVYTLPPSREEMT |
| | (EU numbering | KNQVSLTCLVKGFYPSDIAVEWESNGQPENNYKTTPPMLDSDG |
| | 230-447) | SFFLYSKLTVDKSRWQQGNVFSCSVMHEALHNHYTQKSLSLSP |
| | | GK |
| 33 | Fc(IgG2- | PAPELLGGSSVFLFPPKPKDTLMISRTPEVTCVVVDVSHEDPE |
| | 233E/234L/ | VQFNWYVDGVEVHNAKTKPREEQFNSTFRVVSVLTVVHQDWLN |
| | 235L/236G/ | GKEYKCKVSNKGLPAPIEKTISKTKGQPREPQVYTLPPSREEM |
| | 238S) (EU | TKNQVSLTCLVKGFYPSDIAVEWESNGQPENNYKTTPPMLDSD |
| | numbering | GSFFLYSKLTVDKSRWQQGNVFSCSVMHEALHNHYTQKSLSLS |
| | 230-447) | PGK |
| 34 | Amino acid | GTPTPTPTPTGE |
| | sequence of | |
| | TP linker | |
| 35 | Exemplary | GGGGSGGGGSGGGGS |
| | GS-based | |
| | linker | |
| | sequence | |

### EXAMPLES

### Example 1—Production and Expression of Recombinant Plasminoqen-Fc Fusion

A nucleic acid encoding human plasminogen was cloned into an expression vector at a position 5′ to a sequence encoding a linker and Fc. The nucleotide sequence of the plasminogen-linker-Fc fusion is shown in SEQ ID NO: 21. The linker is GQAGQAS (SEQ ID NO: 23, also referred to herein as the “QA” linker) and the sequence of the Fc encoded is shown in SEQ ID NO: 24. The amino acid sequence of the plasminogen-linker-Fc fusion is shown in SEQ ID NO: 22.

### Materials

Expi293 expression medium (CAT #A1435101), Erlenmeyer flask, Phosphate buffered saline (1×), Glucose (300 mg/mL), polyethylenimine PEI (1 mg/mL), Lupin (125 g/L), Glutamax (100×), pcDNA3.1 Plg (0.5 μg/mL cell culture), pcDNA3.1 PAI-1 (0.5 μg/mL cell culture)

### Method

Day 1: Dilute cells prior to transfection.

Day 2: Add DNA diluted in PBS and PEI to cells. Incubate cells at 37° C. 5% CO₂ at 110-140 rpm.

Days 3-7: Adjust lupin and glucose levels in culture media.

Day 8: Harvest medium by centrifuging culture at 2000×g at 4° C. for 15 minutes.

### Example 2—Purification of Recombinant Plasminoqen-Fc Fusion

For every 100 mL of clarified supernatant, 20 mL of 0.5 M NaH₂PO₄, 5 g of glycerol and 1 Roche protease inhibitor tablet (comprising aprotinin, bestatin, calpain inhibitor I and II, chymostatin, E-64, Leupeptin, pefabloc SC/PMSF, pepstatin, TLCK-HCl, Trypsin inhibitor, Antipain dihydrochloride, phopsphoramidonare) were added and mixed.

Lysine Affinity Column
Buffer A: 100 mM Na₂HPO₄, pH 8.0, 5% glycerol, 0.02% azide Buffer B: 100 mM Na₂HPO₄ pH 8.0, 25 mM EACA (epsilon aminocaproic acid), 5% glycerol

### CV=Column Volume

a) 20 mL Lysine Hyper D resin per 100 mL of culture supernatant.
b) In a gravity-flow column, the resin is washed with 2 CV of MQ H₂O and equilibrated with 2 CV of Buffer A
c) Equilibrated lysine resin is added to clarified media (from step 1) and batch-bound for 1 hour at 4° C.
d) Media allowed to flow-through and flow-through collected;
e) Resin washed with 2 CV buffer A.
f) Elution with buffer B-typically with half resin volume at a time. (For example, 10 mL fractions for 20 mL resin). Bradford's reagent is used to determine elution endpoint.
g) Run fractions on a 10% SDS-PAGE.
h) Pool fractions for next purification step.

HiTrap Q FF
Buffer A: 50 mM Tris pH 9.0, 5 mM EACA, 10% glycerol, 30 mM NaCl, 0.02% azide
Buffer B: 50 mM Tris pH 9.0, 5 mM EACA, 10% glycerol, 1 M NaCl, 0.02% azide
   a) Concentrate sample to 5 ml (50K MWCO), keep pre-column sample and dilute to 50 ml using buffer A
   b) Pre-equilibrate HiTrap Q with 5CV of MQ H₂O and 5CV buffer A
   c) Load sample at 1 ml/min and collect flow-through.
   d) Wash with 5 CV of buffer A
   e) Elution gradient:
      0 to 25% B in 20 CV (100 ml)
      20% to 100% B in 0 CV
      100% for 2CV

Plg is typically eluted as the dominant peak at around 10% B. Run gel to determine purity and pool fractions.

Dialyze pooled fractions overnight at 4° C. in 25 mM Tris pH 7.4, 150 mM NaCl, 5% glycerol. Repeat dialysis for another two hours. This is to ensure the removal of EACA.

### Gel Filtration S200 16/60

Buffer: 25 mM Tris pH 7.4, 150 mM NaCl, 5% glycerol, 1 mM sodium EDTA, 0.02% azide, 1× Roche protease inhibitor cocktail. (Tris can be substituted with Hepes or Na₂HPO₄)
a) Concentrate to at least 5 ml and gel fil on Superdex 200 16/60.
b) Elution volume is around 73 ml.
c) Run gel and pool the relevant fractions.

In the presence of 5% glycerol, plasminogen can be concentrated up to 15 mg/ml in a 50K MWCO concentrator.

Plasminogen can be stored frozen after snap-freezing in liquid N₂.

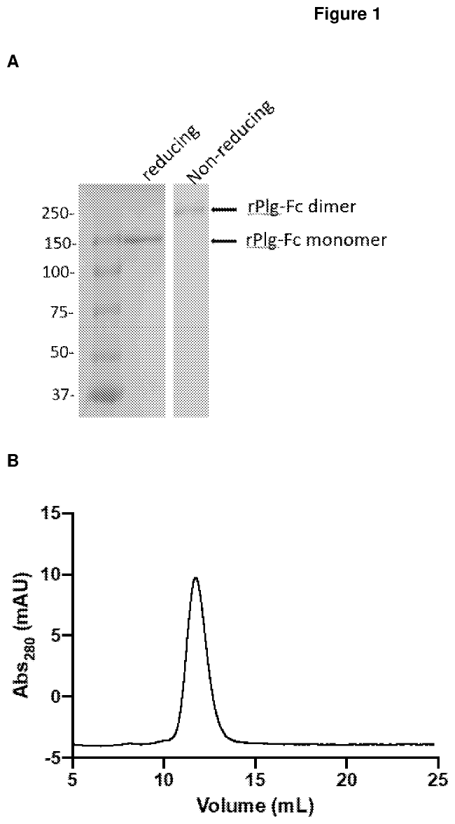

FIG. 1A shows a representative Coomassie stained 10% SDS-PAGE of rPlg-Fc. Protein bands are observed at about 150 kDa under reducing conditions and at about 300 kDa under non-reducing conditions. Expected size without glycosylation: 114.6 kDa. FIG. 1B shows a representative elution profile from a Superdex 200 10/30 column analytical analysis, showing rPlg-Fc is purified as a single species.

Fc-fused mini-pig and micro-pig were also successfully expressed and purified using the methods described herein and shown to be active (data not shown).

### Example 3—Enzyme Mediated Activation of Plasminogen-Fc Fusion

The assay was performed in the presence of 38 nM rPlg-Fc, 7 nM tPA, 100 μM S-2251 (chromogenic substrate), or 100 μM of AFK-AMC (fluorogenic substrate) and 20 mM EACA.

Progression of enzyme activity was monitored at 37° C. using a FLUOstart Omega microplate reader (BMG LABTECH)

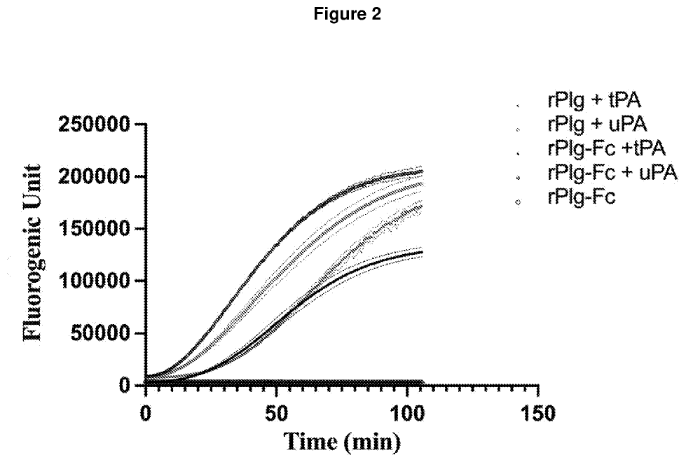

Progress curve showing tPA and uPA-mediated activation of rPlg and rPlg-Fc fusion is shown in FIG. 2. Activation of rPlg and rPlg-Fc was measured by the hydrolysis of plasmin fluorogenic substrate H-Ala-Phe-Lys-AMC. rPlg-Fc alone does not show any hydrolytic activity, as expected. In the presence of tPA or uPA, comparable hydrolytic activity for rPlg and rPlg-Fc is observed.

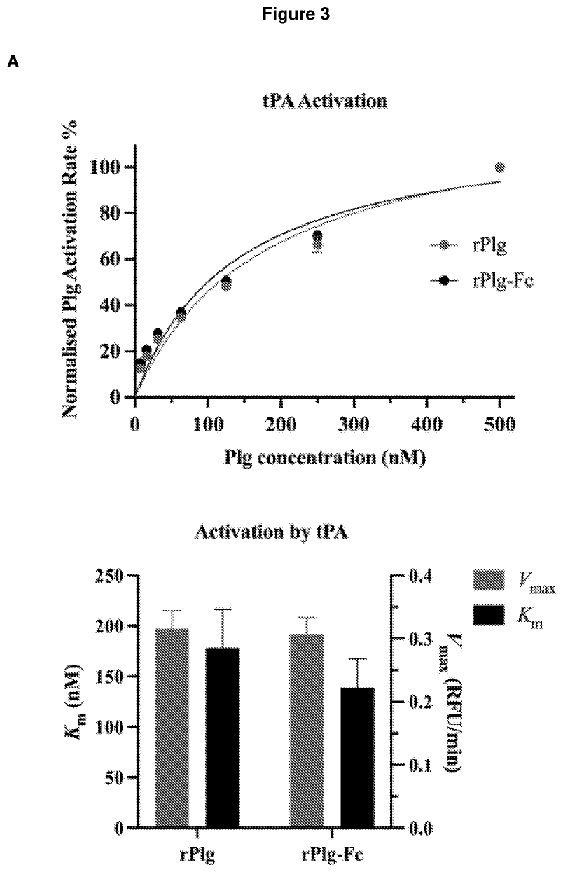

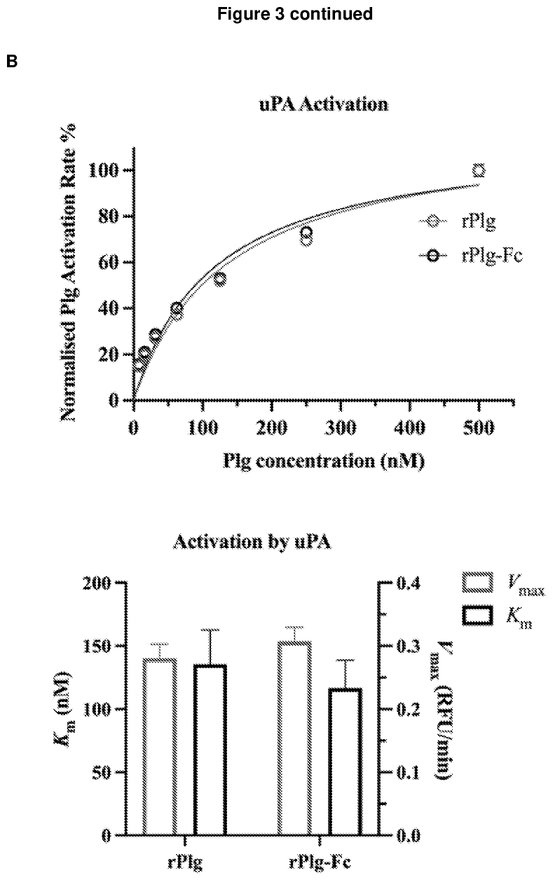

A Michelis-Menten analysis of rPlg and rPlg-Fc activation by tPA and uPA in the presence of 10 mM EACA is shown in FIG. 3. Results from activation of rPlg and rPlg-Fc by tPA (A) and uPA (B) are shown. The result indicates that the kinetics of rPlg and rPlg-Fc activation are similar (as indicated by the KM and Vmax).

### Example 4—Inhibition by α2-Antiplasmin

The recombinant plasminogen-Fc fusion was then analysed for dose dependent α2-antiplasmin inhibition of plasmin activity generated by tPA. A comparison was made between Fc fused plasminogen and non-Fc fused.

The reaction conditions for assay were first performed in 2 steps, first, 20 nM Plg, 10 mM EACA, 100 μM AFK-AMC in the assay buffer was first mixed with A2AP at 0-20 nM. The mixture was incubated at 37° C. for 10 min before tPA was added to 4 nM. The progression of the Plg activation assay was recorded on a BMG Omega Microplate Reader.

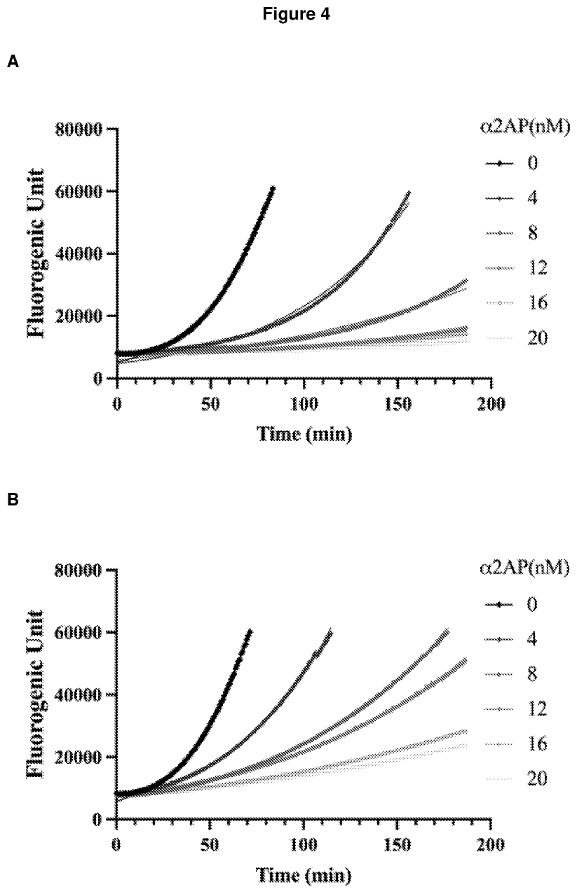

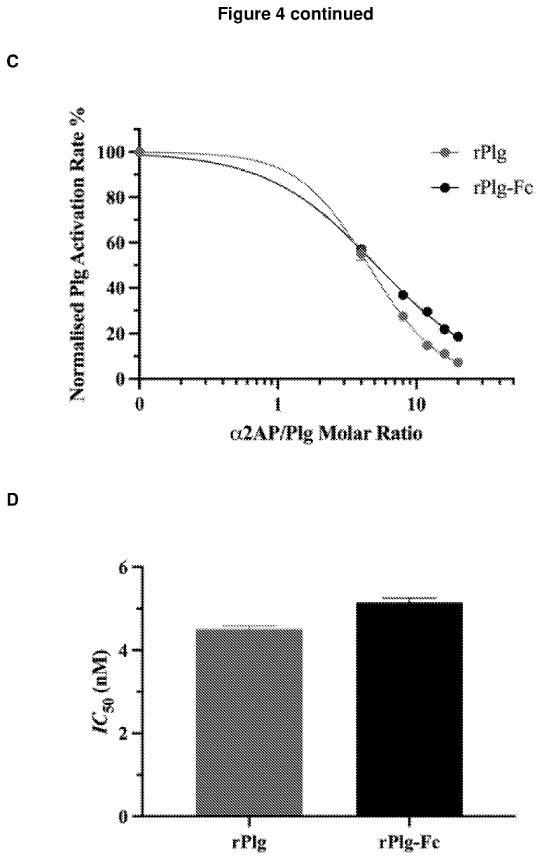

The kinetics of Inhibition by alpha2-antiplasmin (AP) is shown in FIG. 4, specifically a progress curve that was measured in the presence of plasmin fluorogenic substrate H-Ala-Phe-Lys-AMC. It shows a dose dependent inhibition of plasmin activity generated by tPA from rPlg (A) and rPlg-Fc (B). (C) The normalised plasmin activity is plotted against an increasing molar ratio of AP to Plg. The result shows that plasmin generated from rPlg-Fc and rPlg is inhibited by AP. (D) The concentration of AP required to inhibit 50% of plasmin activity (IC₅₀) is derived from (C), and it is comparable for both rPlg and rPlg-Fc.

### Example 5—Storage Stability

The inventors investigated if rPlg-Fc degrades when stored in HBS in the presence of protease inhibitor at different temperatures (37° C., room temperature, 4° C. and −20° C.). The integrity of rPlg-Fc was assessed by SDS-PAGE.

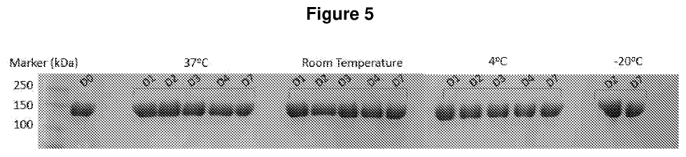

The stability of rPlg-Fc is shown in FIG. 5 at different temperatures. rPlg-Fc was stored at 37° C., room temperature, 4° C. and −20° C. as indicated for up to 7 days (D0-D7) and analysed by Coomassie stained 10% SDS-PAGE under non-reducing conditions. The result showed that no breakdown product is detectable amongst all the samples analysed.

### Example 6—Stability Studies: In Plasma

The inventors investigated if rPlg-Fc degrades upon exposure to plasma at room temperature. To do this rPlg-Fc (with native Plg and rPlg as controls) was labelled with Alexa fluor 647. The integrity of Plg was assessed by fluorescence scanning of SDS-PAGE under reduced and non-reduced conditions (Typhoon5 Phosphoimager/Fluorescence scanner).

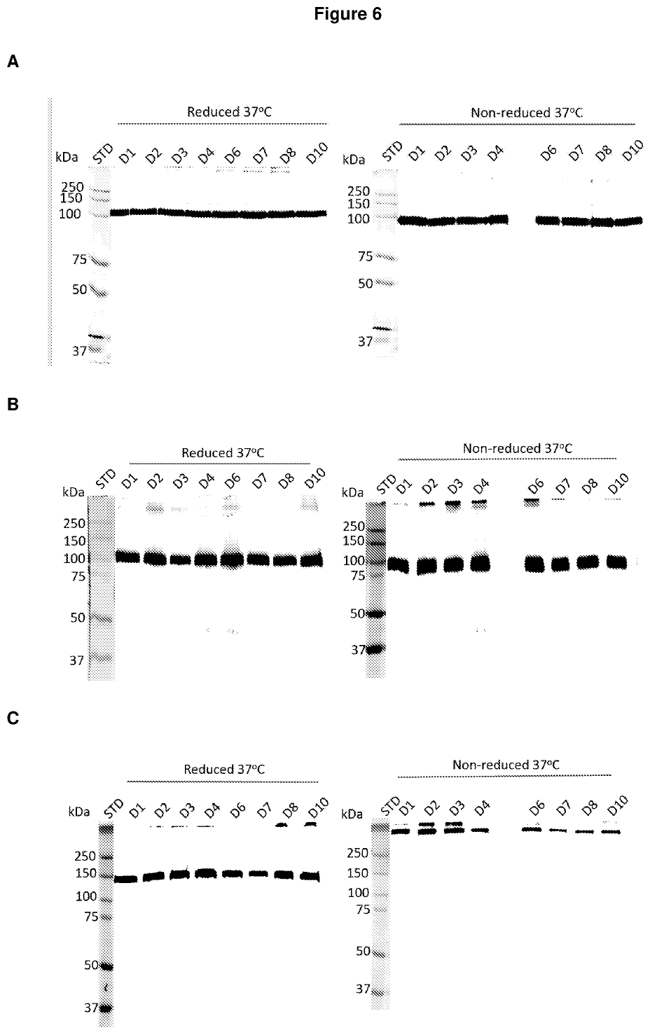

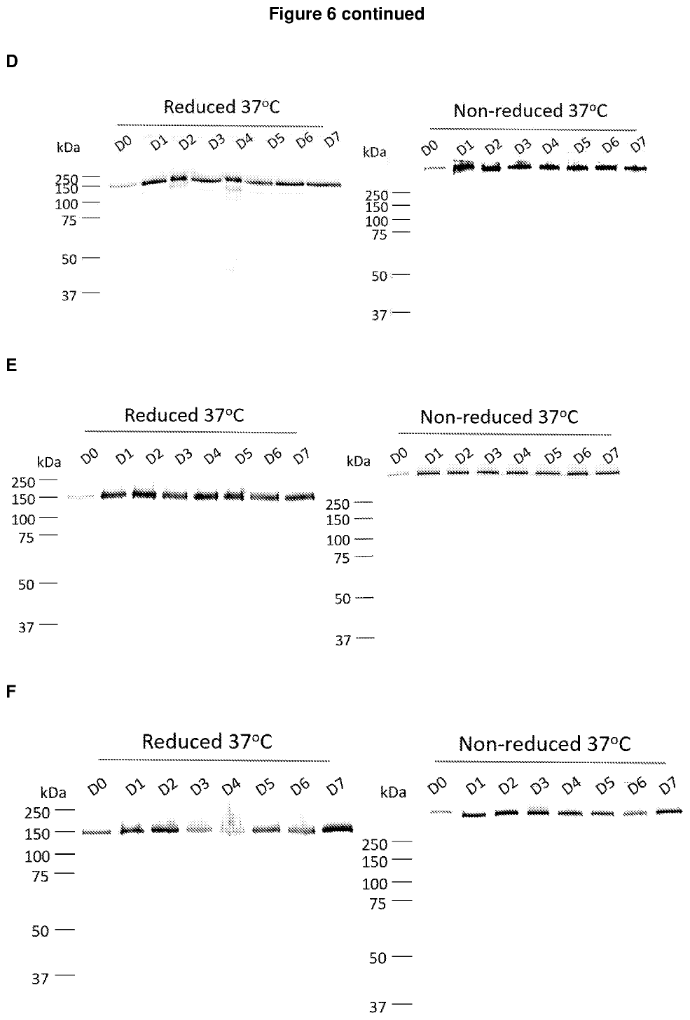

The stability of rPlg-Fc in human plasma is shown in FIG. 6 A-C. Native Plg (A) rPlg (B) and rPlg-Fc (C) was mixed with fresh human plasma from donors @ 2 mM (a physiological concentration) and stored at 37° C. for up to ˜10 days as indicated. Integrity of the proteins was analysed by fluorescence scanning of 10% SDS-PAGE under reducing and non-reducing conditions. The results show that in plasma, no breakdown product is detectable amongst all the samples analysed in this study, suggesting rPlg-Fc is stable in plasma.

A similar experiment was conducted using rPlg-Fc fusion proteins comprising different linker sequences (SEQ ID NOs: 23, 34 and 35), wherein the three different rPlg-Fc fusions were mixed with human plasma freshly collected from donors, at 2 mM and stored at 37° C. for 7 days. The integrity of the proteins was analysed by fluorescence scanning of 10% SDS-PAGE under reducing and non-reducing conditions (FIG. 6D-F). The results show that in plasma, no breakdown product is detectable amongst all the samples analysed.

### Example 7—Stability Study: In Vivo

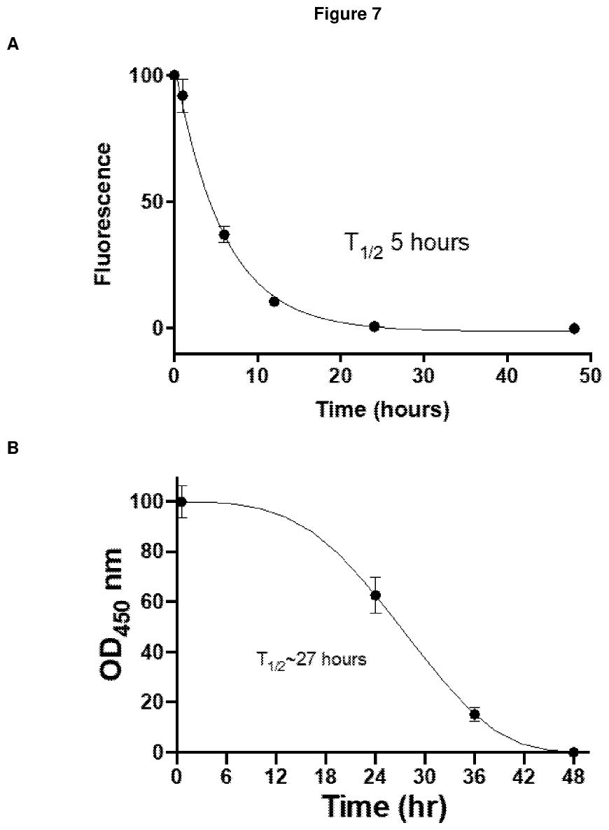

rPlg and rPlg-Fc were injected intravenously at 25 mg/kg; 2 mice were used per time-point. Fluorescently labeled rPlg was used, total fluorescence signal was determined fluorometrically (FIG. 7A). The plasma half-life appears to be just under 5 hours.

Unlabeled rPlg-Fc was used and the amount of full-length molecule in plasma was determined using a sandwich ELISA assay, where a monoclonal anti-Plg antibody was used as a capture antibody and an anti-Fc monoclonal antibody was used as a reporter antibody (FIG. 7B). The plasma half-life appears to be around 27 hours which is about 5 times longer than that of rPlg.

### Example 8—Analysis of Fc Portion Upon Activation

Next the inventors investigated what happens to the Fc portion of the fusion protein upon activation (using either tPA or uPA for 0-120 min in assay buffer). The assay conditions were:
Fc-Plg (in HBS+5% glycerol buffer)
PAs to test: tPA, uPA
Time point:0, 20, 40, 60, 90, 120 min
Buffer: 50 mM Tris pH7.4, 100 mM NaCl, 0.01% Tween-80

| | | |
|---|---|---|
| Component | Activation by tPA | Activation by uPA |

| | | | | |
|---|---|---|---|---|
| Plg stock (1 mg/mL) | 20 | μL | 20 | μL |
| tPA stock (1000 nM) | 2.2 | μL | 0 | μL |

| | | | |
|---|---|---|---|
| uPA stock (500 U/mL) | 0 | 5 | μL |

| | | | | |
|---|---|---|---|---|
| EACA stock (1M) | 1 | μL | 1 | μL |
| Buffer | 77.8 | μL | 75 | μL |
| Total | 100 | μL | 100 | μL |

The reaction was conducted at 30° C. and the reaction mixture was analysed by SDS-PAGE.

| Component | Volume | |
|---|---|---|
| Time Course sample | 2 | μL |
| AFK-AMC (2 mM) | 5 | μL |
| Assay buffer | 93 | μL |
| Total | 100 | μL |

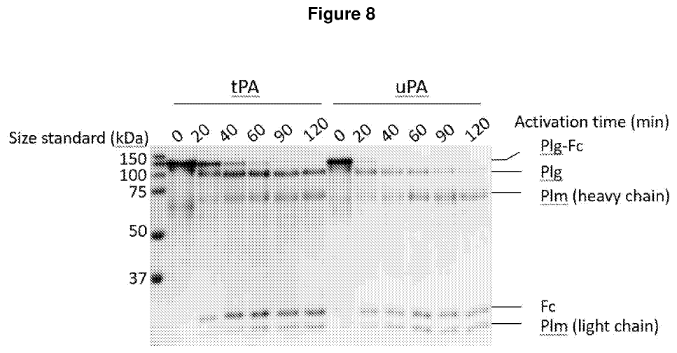

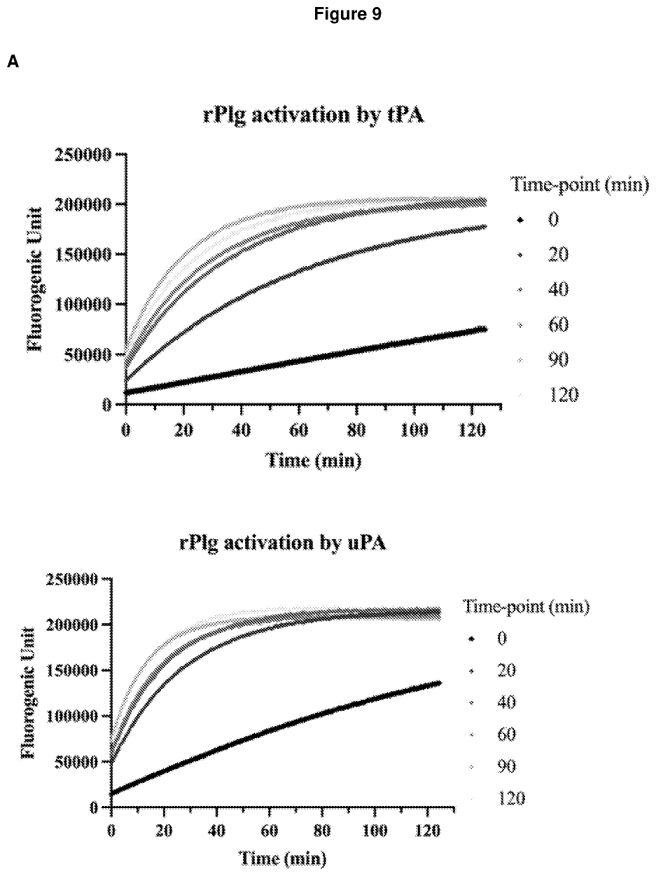

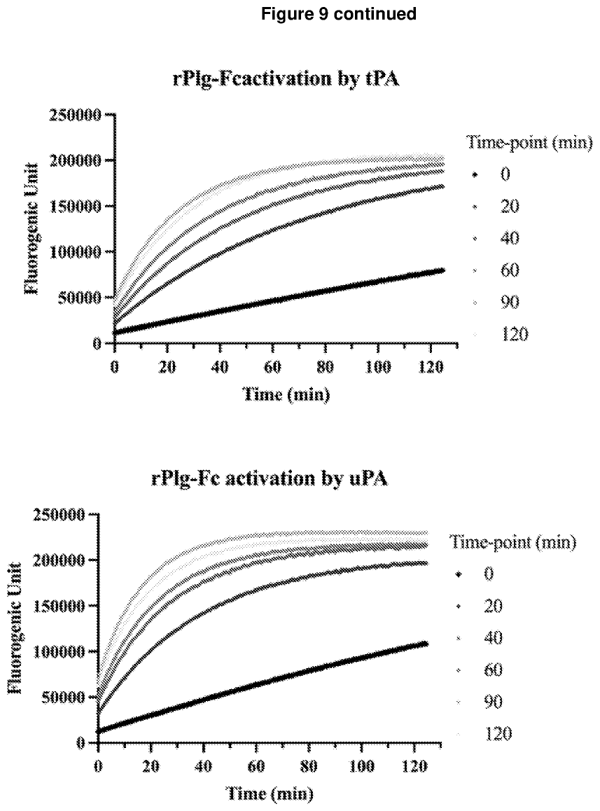

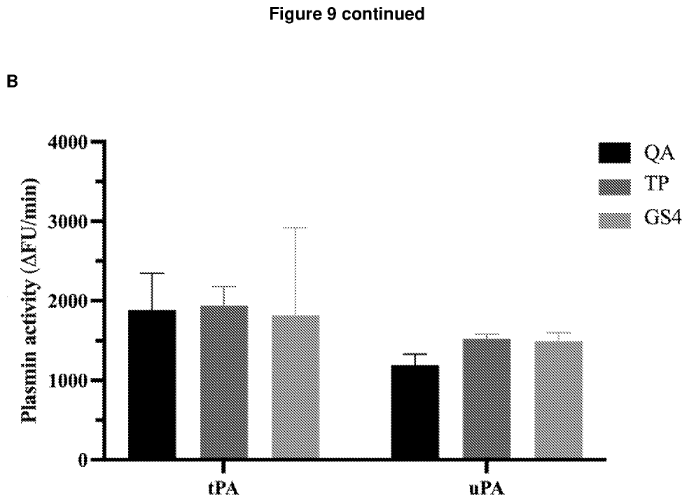

Upon activation in vitro by either tPA or uPA, the Fc portion is cleaved from Plg, as shown in FIGS. 8 and 9. rPlg-Fc was activated by tPA and uPA for up to 120 min as indicated. Samples were analysed by 12% SDS-PAGE followed by Coomassie staining. As shown in FIG. 8, Plg-Fc was quickly cleaved at the linker region generating the Plg and Fc fragments (20 min); Plg is cleaved by the PAs when activated to generate the two chained Plm (heavy and light chains, 20-120 min). uPA is more active as an activator: full-length Plg-Fc is not detected after 40 min; ˜80% of Plg is converted to Plm after 120 min. In the case of tPA: no full-length Plg-Fc is detected after 90 min; ˜50% of Plg is converted to Plm after 120 min. It is not known if the PA or Plm cleaves the linker between Plg and Fc.

Time-course of Plg activation by tPA and uPA is shown in FIG. 9A. rPlg-Fc was activated by tPA and uPA for up to 120 min as indicated. Sample was analysed by Plm activity in the presence of AFK-AMC (Plm fluorogenic substrate), the progression curves are shown and samples as indicated. These data indicate that rPlg-Fc can be activated by the same substrates as rPlg and hPlg and there is no significant difference in activation between rPlg and rPlg-Fc.

The plasmin activity of three different rPlg-Fc proteins, each comprising different linkers, was assessed. Proteins assessed had linkers GQAGQAS (QA), GTPTPTPTPTGE (TP) or GGGGSGGGGSGGGGS (GS).

Following activation with either tPA or uPA, as described above, 4 μL of sample (final concentration of 50 nM), at 40 min was mixed with 5 μL of 2 mM AKF-AMC substrate, 91 μL of assay buffer; enzyme activity was measured at 37° C.

The first 5-6 min of data was analysed within the linear range to calculate the slope in FU/min. As shown in FIG. 9B, the enzyme activity are comparable for all the fusion proteins tested with no significant differences when different linkers are used.

### Example 10—Fibrinolytic Capacity

Synthetic fibrin clots were formed by mixing 3 mg/ml fibrinogen (Banksia Scientific) and 1 U of bovine thrombin (Jomar Life Research) at 37° C. for 2 hours. Fibrinolysis was initiated by addition of 45 nM of plasminogen mixed with 10 nM of tPA (Boehringer Ingelheim) to the surface of the clot.

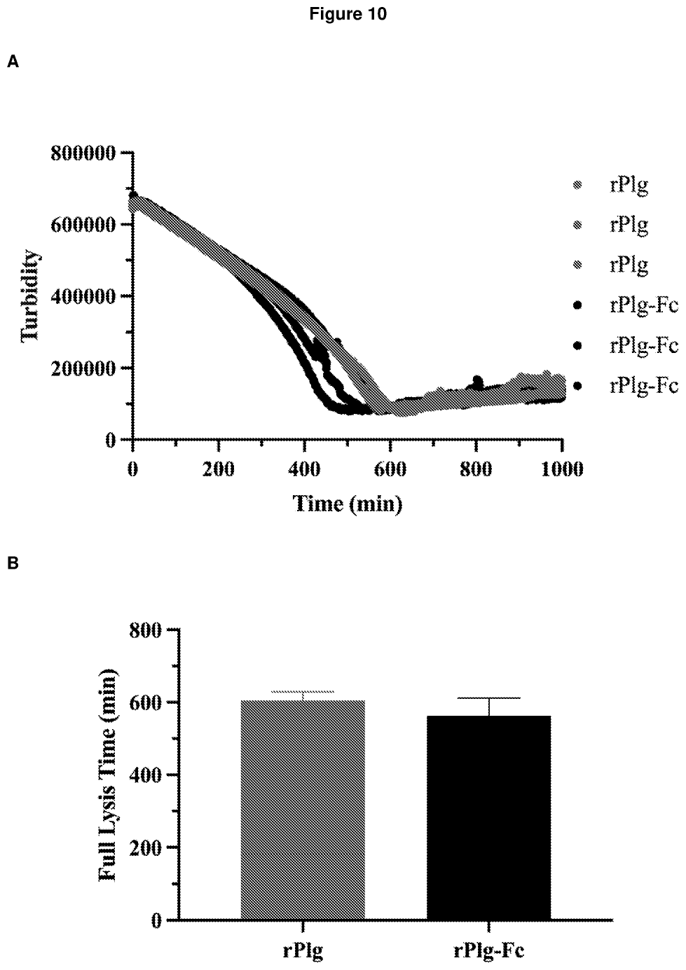

Synthetic clot lysis of rPlg and rPlg-Fc is shown in FIG. 10. The progression curve of clot lysis FIG. 10A was recorded for both rPlg and rPlg-Fc. The time required to achieve full lysis (B) is derived from (A), and it is comparable for rPlg and rPlg-Fc, as shown.

### Example 11—Wound Healing Activity

The therapeutic efficacy of the rPlg-Fc fusion in a diabetic mouse model of wound healing was determined and compared to the following controls: PBS, Fc protein only, rPlg and inactive rPlg (“rPlgRASA” being a form of rPlg without catalytic activity and which cannot be activated due to point mutations S741A and R561A, respectively).

Animals used in the study were 12-14 week old female BKS.Cg-Dock7m^{+/+} Leprdb/J mice. The backs of mice were shaved and 4 full-thickness punch biopsy wounds (5 mm in diameter) were created. At days 1, 3, 5 and 7 following punch biopsy, 1 μm protein was administered to the wounds, intradermally.

Percentage wound closure was assessed at day 11 after punch biopsy. Percentage wound closure was defined as [1−(length of open wound/length of original wound)]×100.

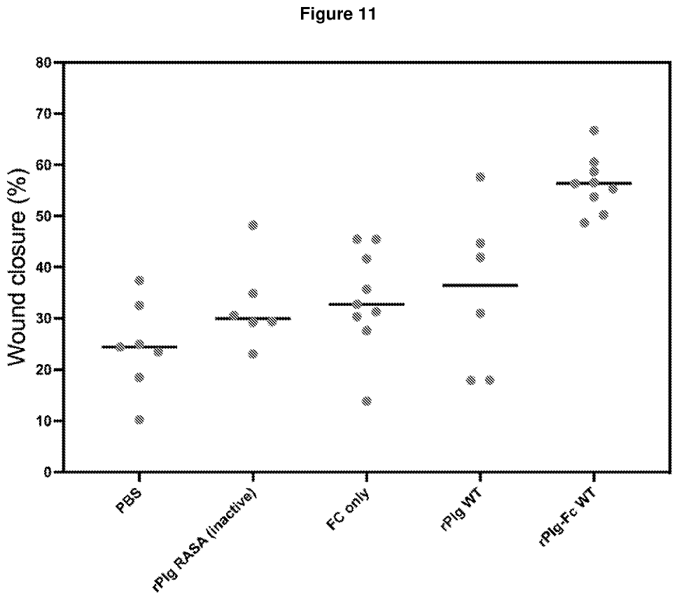

The data shown in FIG. 11 demonstrate that the rPlg-Fc fusion protein is significantly more effective at promoting wound closure compared to non-Fc fused rPlg (as assessed by percentage wound closure).

Overall the data show that rPlg-Fc proteins have greater stability and therapeutic efficacy in vivo compared to rPlg. Further, upon activation, rPlg-Fc has similar activity to rPlg and can be activated by the same substrates as native Plg.

It will be understood that the invention disclosed and defined in this specification extends to all alternative combinations of two or more of the individual features mentioned or evident from the text or drawings. All of these different combinations constitute various alternative aspects of the invention.

1. A chimeric or fusion protein comprising plasminogen and an Fc region of an antibody.

2. The chimeric or fusion protein according to claim 1, wherein the plasminogen corresponds to the plasminogen sequence of a mammal.

3. The chimeric or fusion protein according to claim 1 or 2, wherein the plasminogen is human plasminogen.

4. The chimeric or fusion protein according to claim 1 or 2, wherein the plasminogen is a non-human primate plasminogen, such as pig, mouse, rat, sheep, goat, horse, cow, cat, or dog plasminogen.

5. The chimeric or fusion protein according to any one of claims 1 to 4, wherein the plasminogen is selected from the group consisting of: Glu-Plg, Lys-Plg, Midi-Plg, Mini-Plg and Micro-Plg.

6. The chimeric or fusion protein according to any one of claims 1 to 5, wherein the plasminogen comprises, consists or consists essentially of an amino acid sequence as set forth in any one of SEQ ID NOs: 2, 7, 9, 11, 13, 15, 16, 17, 18, 19 or 20, or a sequence that is at least 75%, at least 80%, at least 85%, at least 90%, at least 91%, at least 92%, at least 93%, at least 94%, at least 95%, at least 96%, at least 97%, at least 98%, at least 99% identical to the amino acid sequence as set forth in any of SEQ ID NOs: 2, 7, 9, 11, 13, 15, 16, 17, 18, 19 or 20.

7. The chimeric or fusion protein according to any one of claims 1 to 6, wherein the plasminogen comprises, consists or consists essentially of an amino acid sequence of SEQ ID NOs: 2, 7, 9, 11, 13, 15, 16, 17, 18, 19 or 20 with 0 to 8 amino acid insertions, deletions, substitutions or additions (or a combination thereof), preferably from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof).

8. The chimeric or fusion protein according to any one of claims 1 to 7, wherein the plasminogen does not contain a signal sequence.

9. The chimeric or fusion protein according to any one of claims 1 to 7, wherein the plasminogen does not contain a signal sequence described herein.

10. The chimeric or fusion protein according to any one of claims 1 to 9, wherein the Fc region of the antibody is an Fc region of an IgG, preferably IgG1.

11. The chimeric or fusion protein according to any one of claims 1 to 10, wherein the Fc region of the fusion protein comprises two heavy chain fragments, more preferably the CH2 and CH3 domains of said heavy chain.

12. The chimeric or fusion protein according to any one of claims 1 to 11, wherein the Fc region of an antibody comprises, consists essentially of or consists of an amino acid sequence of any one of SEQ ID NOs: 24 to 33, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to any one of SEQ ID NOs: 24 to 33.

13. The chimeric or fusion protein according to any one of claims 1 to 12, wherein the Fc region of an antibody comprises, consists essentially of or consists of an amino acid sequence of any one of SEQ ID NOs: 24 to 33 having with 0 to 8 amino acid insertions, deletions, substitutions or additions (or a combination thereof), preferably from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof).

14. The chimeric or fusion protein according to any one of claims 1 to 13, wherein the plasminogen may be covalently linked directly to the Fc region of an antibody.

15. The chimeric or fusion protein according to any one of claims 1 to 14, wherein the plasminogen is separated from the Fc via a linker.

16. The chimeric or fusion protein according to claim 15, wherein the linker comprises or consists of amino acids.

17. The chimeric or fusion protein according to claim 16, wherein the linker comprises or consists of any one or more repeats of glycine and serine.

18. The chimeric or fusion protein according to claim 16, wherein the linker comprises or consists of the amino acid sequence in SEQ ID NO: 23.

19. The chimeric or fusion protein according to any one of claims 1 to 16 or 18, wherein the chimeric or fusion protein comprises or consists of the amino acid sequence in SEQ ID NO: 22.

20. A dimeric protein formed from covalently bonded monomers of the chimeric or fusion protein of any one of claims 1 to 19.

21. The dimeric protein according to claim 20, wherein dimerisation occurs via cysteine residues present in the Fc portion of the chimeric or fusion protein.

22. A nucleic acid comprising or consisting of a nucleotide sequence encoding a chimeric or fusion protein according to any one of claims 1 to 19.

23. The nucleic acid according to claim 22, wherein the nucleotide sequence that encodes a plasminogen comprises, consists or consists essentially of SEQ ID NOs: 1, 5, 6, 8, 10, 12 or 14, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% sequence identity to the sequence set forth in any one of SEQ ID NOs: 1, 5, 6, 8, 10, 12 or 14.

24. A vector or expression construct comprising a nucleic acid according to claim 22 or 23.

25. The vector or expression construct according to claim 24, further comprising a nucleotide sequence encoding PAI-1 or variant thereof, preferably, the nucleotide sequence encoding a chimeric or fusion protein of the invention and nucleotide sequence encoding PAI-1 or variant thereof are operably linked to a promoter for enabling the expression of the polynucleotides.

26. The vector or expression construct according to claim 24, wherein the nucleotide sequence encoding a PAI-1 or variant thereof comprises, consists or consists essentially of the nucleic acid sequence of SEQ ID NO: 3, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% identity to the sequence set forth in SEQ ID NO:3.

27. A host cell comprising a vector or expression construct according to any one of claims 24 to 26.

28. A method for producing a chimeric or fusion protein according to any one of claims 1 to 20, the method comprising:
(i) providing a host cell comprising a first nucleic acid encoding a chimeric or fusion protein according to any one of claims 1 to 19 and a second nucleic acid encoding a plasminogen activator inhibitor, preferably plasminogen activator inhibitor-1 (PAI-1) or variant thereof;
(ii) culturing said host cell in a suitable culture medium under conditions to effect expression of the chimeric or fusion protein from the first nucleic acid and plasminogen activator inhibitor from the second nucleic acid.

29. A method of producing a chimeric or fusion protein according to any one of claims 1 to 20, the method comprising the steps of:
(a) providing a first nucleic acid encoding a chimeric or fusion protein according to any one of claims 1 to 19,
(b) providing a second nucleic acid encoding PAI-1 or variant thereof;
wherein the first and the second nucleic acids are operably linked to a promoter for enabling the expression of the nucleic acids encoding the chimeric or fusion protein of the invention and PAI-1 or variant thereof,
(c) providing a host cell,
(d) transforming or transfecting the host cell with the nucleic acids of a) and b)
(e) providing cell culture media,
(f) culturing the transformed or transfected host cell in the cell culture media under conditions sufficient for expression of the nucleic acids encoding the chimeric or fusion protein and the PAI-1 or variant thereof, and
optionally (g) recovering or purifying the chimeric or fusion protein from the host cell and/or the cell culture media.

30. An isolated, purified, substantially purified or recombinant chimeric or fusion protein produced by a method according to claim 28 or 29.

31. A composition comprising a chimeric or fusion protein according to any one of claims 1 to 21, nucleic acid according to claim 22, a vector or expression construct according to any one of claims 24 to 26, a host cell according to claim 27, and a pharmaceutically or physiologically acceptable carrier, diluent or excipient.

32. A composition comprising a chimeric or fusion protein according to any one of claims 1 to 21 and plasminogen activator inhibitor.

33. The composition according to claim 32 wherein the plasminogen activator inhibitor is PAI-1 or a functionally equivalent variant thereof.

34. A method of treating a condition in an individual, wherein the condition requires administration of exogenous plasminogen (or plasmin), the method comprising administering an isolated, purified, substantially purified or recombinant chimeric or fusion protein according to any one of claims 1 to 21, or chimeric or fusion protein comprising plasmin derived therefrom, to the individual, thereby treating the condition requiring administration of exogenous plasminogen (or plasmin),

35. A method of inducing or promoting lysis of a pathological fibrin deposit in a subject, comprising administering a chimeric or fusion protein according to any one of claims 1 to 21, nucleic acid according to claim 22, a vector or expression construct according to any one of claims 24 to 26, a host cell according to claim 27 to the subject, thereby inducing or promoting lysis of a pathological fibrin deposit in the subject.

36. Use of a chimeric or fusion protein according to any one of claims 1 to 21, nucleic acid according to claim 22, a vector or expression construct according to any one of claims 24 to 26, a host cell according to claim 27 in the manufacture of a medicament for inducing or promoting lysis of a pathological fibrin deposit in a subject.
